(19) United States
(12) Patent Application Publication
(10) Pub. No.: US 2026/0242347 A1
(43) Pub. Date: Aug. 20, 2026

(54) Analogs of Cannabichromene and Cannabichromenic Acid and Process of Preparing

(71) Applicant: Bessor Pharma, LLC, Framingham, MA (US)

(72) Inventors: Barry A. Berkowitz, Framingham, MA (US); Anthony G. Barrett, Rio de Janeiro (BR); Daniel Elliott, Basel (CH)

(21) Appl. No.: 19/164,863

(22) PCT Filed: Mar. 15, 2024
(86) PCT No.: PCT/US2024/020086

§ 371 Date: Sep. 12, 2025

Related U.S. Application Data
(60) Provisional application No. 63/490,402, filed on Mar. 15, 2023.

(51) Int. Cl. C07D 311/58; C07D 311/70; C07D 493/04

(52) U.S. Cl. CPC C07D 311/58; C07D 311/70; C07D 493/04

(57) ABSTRACT

Novel cannabinoids cannabichromene (CBC, 1), cannabichromenic acid (CBCA, 2), cannabichromevarin (CBCV, 3), cannabichromevarinic acid (CBCVA, 4) and other chromene cannabinoids 7 as racemic modifications, as single enantiomers (S)-7 or (R)-7 or a scalemic mixtures of (S)-7 and (R)-7 are analogs of cannabichromene (CBC, 1), cannabichromenic acid (CBCA, 2), cannabichromevarin (CBCV, 3) and cannabichromevarinic acid (CBCVA, 4) all as racemic compounds, single enantiomers and as scalemic mixtures. In addition, the present invention also relates to novel key intermediates of the formulae 9 to 15 for the synthesis of the cannabinoids above.

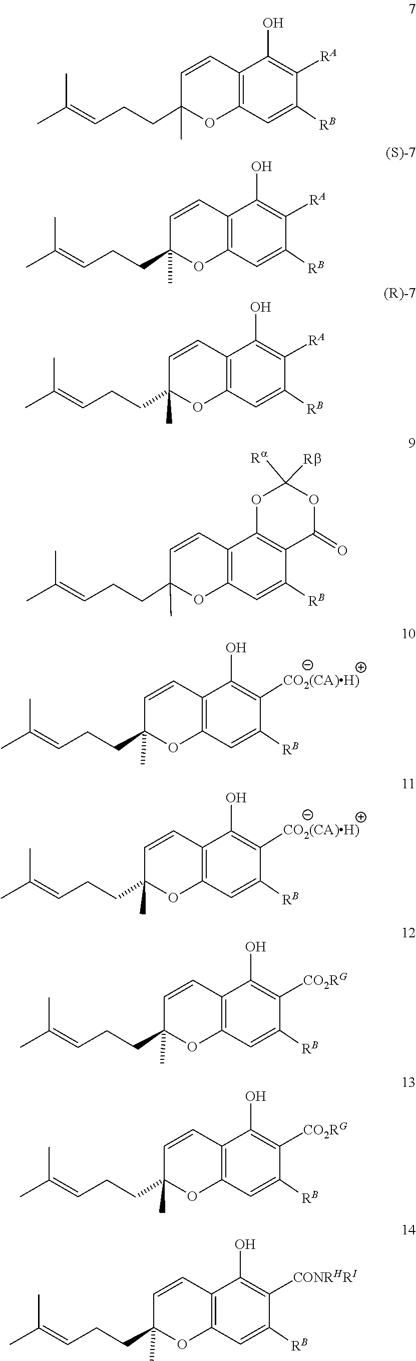

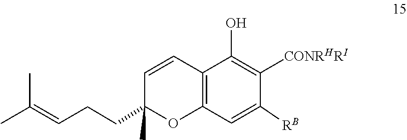

### FIELD OF THE INVENTION

The field of the invention relates to methods for the synthesis of high purity known and novel cannabinoids including but not limited to cannabichromene (CBC, 1), cannabichromenic acid (CBCA, 2), cannabichromevarin (CBCV, 3), cannabichromevarinic acid (CBCVA, 4) and analogous cannabinoids and other synthetic analogues from simple inexpensive starting materials by construction of the aromatic core. The field of the invention also includes methods for the resolution of cannabichromene (CBC, 1), cannabichromenic acid (CBCA, 2), cannabichromevarin (CBCFV, 3), cannabichromevarinic acid (CBCVA, 4) and related chromene cannabinoids and cannabinoid carboxylate acids by direct separation of the enantiomers by HPLC, or by formation of diastereoisomeric co-crystals and their separation and reconversion to the resolved cannabinoid, or by formation of diastereoisomeric salts from cannabichromenic acid (CBCA, 2), cannabichromevarinic acid (CBCVA, 4) and related cannabinoid acids with chiral amines and the conversion of these salts with alkali or base and subsequent acidification with optional decarboxylation into single enantiomer cannabichromene (CBC, 1), cannabichromenic acid (CBCA, 2), cannabichromevarin (CBCV, 3), cannabichromevarinic acid (CBCVA, 4) and analogous cannabinoids of both enantiomeric series as well as scalemic mixtures of enantiomers. The field of the invention additionally covers novel cannabinoids, which may be used as active compounds either alone or admixed in combination with known cannabinoids, terpenes or other drugs in drug formulations for the treatment or prevention of infections, pain, multiple sclerosis-related spasticity, nausea, anorexia, epilepsy, Alzheimer's and neurdegenerative diseases, brain injury/concussion/traumatic brain injury, stroke, cancer, reduction of inflammation and immuno-inflammation related diseases, Post viral hyperinflammation, diseases/injury of the eye including but not limited to glaucoma, dry eye, corneal injury or disease and retinal degeneration or disease, disorders of immune-inflammation, lung injury or disease, liver injury or disease, kidney injury or disease, pancreatitis and disorders of the pancreas cardiovascular injury or disease, and organ transplant, reduction of post-surgical inflammation among other diseases, and as anti-oxidants, diseases of the skin.

### BACKGROUND OF THE INVENTION

Cannabis sativa (“marijuana”) is a plant of considerable notoriety and use. Marijuana or cannabis use as a recreational drug worldwide, has been and remains the subject of legal review in many countries of the world. There has been very considerable interest in the use of this plant and its extracts as ethnopharmaceuticals for millennia with reference even in Herodotus, (The Histories, Book IV, page 295, Penguin Books, Ltd., Middlesex (1972). The plant and its extracts have been used in medicine on account of their effects as anesthetics, spasmolytics, and hypnotic agents, immune-inflammation regulatory agents to combat the side effects of nausea following cancer chemotherapy, in the treatment of glaucoma, neuropathic pain, epilepsy, multiple sclerosis-related spasticity and pain in patients with advanced cancer, AIDS-related anorexia and pain.

There are over 60 constituent compounds that have been isolated and characterized from Cannabis sativa oil (for example see S. A. Ahmed, S. A. Ross, D. Slade, M. M. Radwan, F. Zulfiqar and M. A. ElSohly “Cannabinoid Ester Constituents from High-Potency Cannabis sativa”, Journal of Natural Products, 2008, volume 71, pages 536-542; M. M. Lewis, Y. Yang, E. Wasilewski, H. C. Clarke and L. P. Kotra, “Chemical Profiling of Medical Cannabis Extracts”, ACS Omega, 2017, volume 2, pages 6091-6103 and references therein). In addition, a considerable number of these natural products and analogs have been prepared by total synthesis from aromatic and monoterpene precursor compounds. Such total syntheses are reported (for examples see R. K. Razdan, “The Total Synthesis of Cannabinoids” in “The Total Synthesis of Natural Products”, Editor J. ApSimon, 1996, volume 4, pages 185-262, New York, N. Y.: Wiley and Sons; J. W. Huffman and J. A. H. Lainton, “Recent Developments in the Medicinal Chemistry of Cannabinoids”, Current Medicinal Chemistry, 1996, volume 3, pages 101-116; N. Itagaki, T. Sugahara and Y. Iwabuchi, “Expedient Synthesis of Potent Cannabinoid Receptor Agonist (−)-CP55,940”, Organic Letters, 2005, volume 7, pages 4181-4183; J. A. Teske and A. Deiters, “A Cyclotrimerization Route to Cannabinoids”, Organic Letters, 2008, volume 10, pages 2195-2198; S. Tchilibon and R. Mechoulam, “Synthesis of a Primary Metabolite of Cannabidiol”, Organic Letters, 2000, volume 2, pages 3301-3303; Y. Song, S. Hwang, P. Gong, D. Kim and S. Kim*, “Stereoselective Total Synthesis of (−)-Perrottetinene and Assignment of Its Absolute Configuration”, Organic Letters, 2008, volume 10, pages 269-271; Y. Kobayashi, A. Takeuchi and Y.-G. Wang, “Synthesis of Cannabidiols via Alkenylation of Cyclohexenyl Monoacetate”, Organic Letters, 2006, volume 8, pages 2699-2702; B. M. Trost and K. Dogra, “Synthesis of (−)-Δ⁹-trans-Tetrahydrocannabinol: Stereocontrol via Mo-Catalyzed Asymmetric Allylic Alkylation Reaction”, Organic Letters, 2007, volume 9, pages 861-863; L.-J. Cheng, J.-H. Xie, Y. Chen, L.-X. Wang and Q.-L. Zhou, “Enantioselective Total Synthesis of (−)-Δ⁸-THC and (−)-Δ⁹-THC via Catalytic Asymmetric Hydrogenation and S_{N}Ar Cyclization” Organic Letters, 2013, volume 15, pages 764-767; P. R. Nandaluru and G. J. Bodwell, “Multicomponent Synthesis of 6H-Dibenzo[b,d]pyran-6-ones and a Total Synthesis of Cannabinol”, Organic Letters, 2012, volume 14, pages 310-313; S. Ben-Shabat, LO. Hanus, G. Katzavian and R. Gallily, “New Cannabidiol Derivatives: Synthesis, Binding to Cannabinoid Receptor, and Evaluation of Their Antiinflammatory Activity”, Journal of Medicinal Chemistry, 2006, volume 49, pages 1113-1117; A. Mahadevan, C. Siegel, B. R. Martin, M. E. Abood, I. Beletskaya and R. K. Razdan, “Novel Cannabinol Probes for CB1 and CB2 Cannabinoid Receptors”, Journal of Medicinal Chemistry, 2000, volume 43, pages 3778-3785; S. P. Nikas, S. O. Alapafuja, I. Papanastasiou, C. A. Paronis, V. G. Shukla, D. P. Papahatjis, A. L. Bowman, A. Halikhedkar, X. Han and A. Makriyannis, “Novel 1′,1′-Chain Substituted Hexahydrocannabinols: 9β-Hydroxy-3-(1-hexyl-cyclobut-1-yl)-hexahydrocannabinol (AM2389) a Highly Potent Cannabinoid Receptor 1 (CB1) Agonist”, Journal of Medicinal Chemistry, 2010, volume 53, pages 6996-7010; M. J. Kavarana and R. C. Peet, “Bioenzymatic Synthesis of THC-V, CBV and CBN and their use as Therapeutic Agents”, US Patent Application, 2017/0283837 A1; R. Winnicki, M. Donsky, M. Sun and R. Peet, “Apparatus and Methods for Biosynthetic Production of Cannabinoids’, U.S. Pat. No. 9,879,292 B2; P. D. Giorgi, V. Liautard, M. Pucheault and S. Antoniotti, “Biomimetic Cannabinoid Synthesis Revisited: Batch and Flow All-Catalytic Synthesis of (±)-ortho-Tetrahydrocannabinols and Analogues from Natural Feedstocks”, European Journal of Organic Chemistry, 2018, pages 1307-1311; S. Morimoto, K. Komatsu, F. Taura and Y. Shoyama, “Enzymological Evidence for Cannabichromenic Acid Biosynthesis”, Journal of Natural Products, 1997, volume 60, pages 854-857; H. Saimoto, K. Yoshida, T. Murakami, M. Morimoto, H. Sashiwa and Y. Shigemasa, “Effect of Calcium Reagents on Aldol Reactions of Phenolic Enolates with Aldehydes in Alcohol”, The Journal of Organic Chemistry, 1996, volume 61, pages 6768-6769; F. Pollastro, D. Caprioglio, P. Marotta, A. S. Moriello, L. De Petrocellis, O. Taglialatela-Scafati and G. Appendino, “Iodine-Promoted Aromatization of p-Menthane-Type Phytocannabinoids”, Journal of Natural Products, 2018, volume 81, pages 630-633; K. P. Bastola, A. Hazekamp and R. Verpoorte, “Synthesis and Spectroscopic Characterization of Cannabinolic Acid”, Planta Medica, 2007, volume 73, pages 273-275).

In the last twenty years it has become apparent that the cannabinoids are in a renaissance for diverse biomedical uses. The pharmacology of the cannabinoids has been shown to be associated with a number of receptors and mechanisms including cannabinoids receptors, GPCR receptors, serotonin receptors, modulation of several voltage-gated channels (including Ca²⁺, Na⁺, and various type of K⁺ channels), ligand-gated ion channels (i.e., GABA, glycine and TRPV), Toll like receptors, opioid receptors, NMDA or excitatory amino acids receptors, catecholamine receptors, enzymes regulating endocannabinoids, and ion-transporting membranes proteins such as transient potential receptor class (TRP) channels (L. De Petrocellis, M. Nabissi, G. Santoni and A. Ligresti, “Actions and Regulation of Ionotropic Cannabinoid Receptors”, Advances in Pharmacology, 2017, volume 80, pages 249-289; P. Morales and P. H. Reggio, “An Update on Non-CB₁, Non-CB₂ Cannabinoid Related G-Protein-Coupled Receptors”, Cannabis Cannabinoid Research, 2017, volume 2, pages 265-273). Thus, it would be helpful to have a new medicament or medicaments that include one or more cannabinoids for treatment of afflictions known to be treatable by affecting or using these physiological mechanisms.

The pharmacology of the cannabinoids is directly or indirectly receptor-mediated for example, by two G protein-coupled receptors, named CB₁ and CB₂, which have 44% sequence homology in humans. The CB₁ sub-type is the most widely expressed G protein-coupled receptor in the brain in regions, for example, that control motor, emotional, cognitive, sensory responses, perception of pain, thermoregulation, as well as cardiovascular, gastrointestinal, and respiratory physiology. It is localized in the central (CNS) and peripheral nervous systems including the olfactory bulb, cortical areas, parts of the basal ganglia, thalamus, hypothalamus, cerebellar cortex, brainstem, and spinal cord. CB₁ receptors also occur in cells in the pituitary and thyroid glands, some fat, muscle and liver cells as well as the lung and kidneys. The CB₂ sub-type is expressed in immune and hematopoietic cells, osteoclasts, and osteoblasts and mediates the response of the immune system, controls inflammation, modulates inflammatory and neuropathic pain as well as bone remodeling.

The pharmacology of modulators of CB₁ and CB₂ receptors has been reviewed for example by Vemuri and Makriyannis (V. K. Vemuri and A. Makriyannis, “Medicinal Chemistry of Cannabinoids”, Clinical Pharmacology & Therapeutics, 2015, volume 97, pages 553-558). The psychoactive effects of Δ⁹-tetrahydrocannabinol (THC) as well as with its primary metabolite 11-hydroxy-Δ⁹-tetrahydrocannabinol are mediated by its partial agonism of CNS CB₁ receptors (J. van Amsterdam, T. Brunt and W. van den Brink, “The adverse health effects of synthetic cannabinoids with emphasis on psychosis-like effects”, Journal of Psychopharmacology, 2015, volume 29, pages 254-263; R. G. Pertwee, “The diverse CB₁ and CB₂ receptor pharmacology of three plant cannabinoids: Δ⁹-tetrahydrocannabinol, cannabidiol and Δ⁹-tetrahydrocannabivarin”, British Journal of Pharmacology, 2008, volume 153, pages 199-215). It is useful as an analgesic, an antiemetic agent, and for treating anorexia in patients with AIDS. Other CB₁ receptor modulators include tetrahydrocannabivarin (THCV) (weak antagonist) and cannabinol (CBN) (weak agonist) and both are modest agonists of CB₂. Both the non-psychoactive (−)-cannabidiol (CBD) and cannabidivarin (CBDV) do not interact significantly with either receptor sub-class and their modes of action are less clear (J. Fernãndez-Ruiz, O. Sagredo, M. R. Pazos, C. García, R. Pertwee, R. Mechoulam, J. Martínez-Orgado, “Cannabidiol for neurodegenerative disorders: important new clinical applications for this phytocannabinoid?”, British Journal of Clinical Pharmacology, 2013, volume 75, pages 323-333; S. Rosenthaler, B. Pöhn, C. Kolmanz, C. N. Huu, C. Krewenka, A. Huber, B. Kranner, W.-D. Rausch and R. Moldzio, “Differences in receptor binding affinity of several phytocannabinoids do not explain their effects on neural cell cultures”, Neurotoxicology and Teratology, 2014, volume 46, pages 49-56). The combination of Δ⁹-tetrahydrocannabinol (THC) and cannabidiol (CBD) (Sativex, Nabiximols) is used to treat multiple sclerosis-related spasticity and as a potent analgesic in patients with advanced stage cancers. More recently, purified cannabidiol (CBD) was granted orphan drug status for treating epilepsy. CB₁ receptor antagonists are appetite suppressants, enhance cognition, and control addictive behavior. Selective CB₂ agonists may provide superior analgesic agents and immunomodulators that do not have the undesirable psychoactive effects associated with CNS CB₁ agonism. Δ⁹-tetrahydrocannabinol (THC) (Dronabinol) has been shown to be clinically effective either in monotherapy or in combination with ondansetron (Zofran, a 5-HT₃ antagonists) and in combination with prochlorperazine (a dopamine D₂ receptor antagonist) to treat chemotherapy-induced nausea and vomiting in cancer patients (M. B. May and A. E Glode, “Dronabinol for chemotherapy-induced nausea and vomiting unresponsive to antiemetics”, Cancer Management and Research, 2016, volume 8, pages 49-55).

Cannabinoids that are used as therapeutics are primarily obtained from the extraction of cannabis plant materials, fractionation of Cannabis sativa oil or from total synthesis usually from aromatic and terpene starting materials. Since there are over 60 different natural products in cannabis oil, such oil fractionation requires extensive chromatographic purification to provide any individual constituent substantially pure (>99% pure) and, with so many components, makes reproducible production and storage difficult. For example, the purification of Δ⁹-tetrahydrocannabinol (THC) from other cannabis constituents but particularly from its isomer Δ⁸-tetrahydrocannabinol is inefficient and costly. In addition, since many of the cannabinoids in cannabis oil have different effects as total, partial, inverse or neutral agonists or antagonists of either or both of the CB₁ and CB₂ receptors, it is especially important that individual isolated natural products do not contain significant levels (below parts per million levels) of any other cannabinoid natural product, which has undesired biological effects and that the specifications set are efficiently reproducible. There is an added complication in that many cannabinoid natural products are obtained as oils, which are typically not possible to crystallize and which are prone to air oxidative degradation and their isolation requires the use of extensive expensive and difficult to scale chromatography and/or derivatisation (for example see B. Trawick and M. H. Owens, “Process for the Preparation of (−)-delta 9-Tetrahydrocannabinol”, WO 2009/099868 A1; E. Arslantas and U. Weigl, “Method for Obtaining Pure Tetrahydrocannabinol”, U.S. Pat. No. 7,923,558 B2; J. E. Field, J. Oudenes, B. I. Gorin, R. Orprecio, F. E. Silva e Souza, N. J. Ramjit and E.-L. Moore, “Separation of Tetrahydrocannabinols”, U.S. Pat. No. 7,321,047 B2; P. Bhatarah, K. J. Batchelor, D. McHattie and A. K. Greenwood, “Delta 9 Tetrahydrocannabinol Derivatives”, WO 2008/099183 A1; D. C. Burdick, S. J. Collier, F. Jos, B. Biolatto, B. J. Paul, H. Meckler, M. A. Helle and A. J. Habershaw, “Process for Production of Delta-9-Tetrahydrocannabinol”, U.S. Pat. No. 7,674,922 B2).

The cannabinoids cannabichromene (CBC, 1), cannabichromenic acid (CBCA, 2), cannabichromevarin (CBCV, 3) and cannabichromevarinic acid (CBCVA, 4) have also been isolated and characterized from Cannabis sativa oil in variable purities. Cannabichromene (CBC, 1) is one of most abundant constituents of cannabis oil.

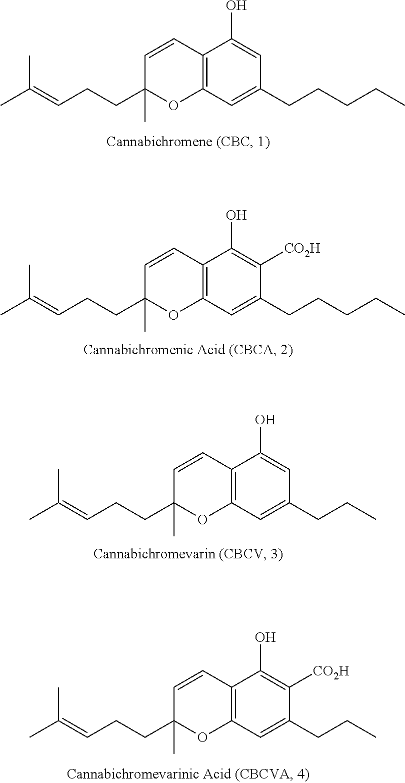

Many of the known synthetic routes to prepare cannabinoids either use expensive reagents and are uneconomic to use on a large scale or are dependent on the condensation reactions of monoterpene starting materials with derivatives of alkyl-resorcinol such as 5-n-pentyl-resorcinol (olivetol) under acidic reaction conditions, reactions that frequently give rise to side products derived from carbenium ion rearrangement reactions and/or side reactions. For example, the manufacture of Δ⁹-tetrahydrocannabinol (THC) from olivetol and monoterpenes by Brønsted or Lewis acid catalyzed condensation reactions is complicated by the co-formation of its isomer Δ⁸-tetrahydrocannabinol, amongst other impurities. Such impurities also considerably complicate and increase the cost of obtaining cannabinoid active pharmaceutical ingredients substantially pure (for examples see R. K. Razdan, “The Total Synthesis of Cannabinoids” in “The Total Synthesis of Natural Products”, Editor J. ApSimon, 1996, volume 4, pages 185-262, New York, N. Y.: Wiley and Sons; C. Steup and T. Herkenroth, “Process for Preparing Synthetic Cannabinoids”, US Patent Application 2010/0298579 A1; R. J. Kupper, “Cannabinoid Active Pharmaceutical Ingredient for Improved Dosage Forms”, WO 2006/133941 A2; J. Erler, and S. Heitner, “Method for the Preparation of Dronabinol”, U.S. Pat. No. 8,324,408 B2; A. L. Gutman, M. Etinger, I. Fedotev, R. Khanolkar, G. A. Nisnevich, B. Pertsikov, I. Rukhman and B. Tishin, “Methods for Purifying trans-(−)-Δ⁹-Tetrahydrocannabinol and trans-(+)-Δ⁹-Tetrahydrocannabinol”, U.S. Pat. No. 9,278,083 B2). Cannibichromene (CBC, 1) and related chromenes have previously been synthesized on a small-scale using an oxidative cyclization reaction with 2,3-dichloro-5,6-dicyano-p-benzoquinone in benzene or by amine-catalyzed cyclocondensation reactions of 5-alkyl-resorcinols with citral (G. Cardillo, R. Cricchio and L. Merlini, “Synthesis of D,L-Cannabichromene, Franklinone and other Natural Chromenes, Tetrahedron, 1968, volume 24, pages 4825-4831) or 1,4-dioxane (K. Morimoto, S. Taura, “Enzymological Evidence for Cannabichromenic Acid Biosynthesis”, Journal of Natural Products, 1997, volume 60, pages 854-867; T. Hashimoto, D. N. Quang, M. Nukada, Y. Asakawa, “Isolation, Synthesis and Biological Activity of Grifolic Acid Derivatives from the Inedible Mushroom Albatrellus dispansus”, Heterocycles, 2005, volume 65, pages 2431-2439; Y. Shoyama, H. Hirano, I, Nishioka, “Biosynthesis of propyl cannabinoid acid and its biosynthetic relationship with pentyl and methyl cannabinoid acids”, Phytochemistry, 1984, volume 23, pages 1909-1912), or with dichromate salts (G. Cardillo, M. Orena, G. Porzi, S. Sandri, “Simple Methods for Oxidation of o-Allylphenols to Chrom-3-enes”, Journal of the Chemical Society, Chemical Communications, 1979, pages 836-837) or using N-iodosuccinimide (A. Bongini, G. Cardillo, M. Orena, G. Porzi and S. Sandri, “A New Synthesis of 2,2′-Dialkylchrom-3-enes and Flav-3-enes”, Tetrahedron Letters, 1979, pages 2545-2548; G.-N. Nguyen, E. N. Jordan and O. Kayser, “Synthetic Strategies for Rare Cannabinoids Derived from Cannabis sativa”, Journal of Natural Products, 2022, volume 85, pages 1555-1568; L. L. Anderson, A. Ametovski, J. L. Luo, D. Everett-Morgan, I. S. McGregor, S. D. Banister and J. C. Arnold, “Cannabichromene, Related Phytocannabinoids, and 5-Fluoro-cannabichromene Have Anticonvulsant Properties in a Mouse Model of Dravet Syndrome”, ACS Chemical Neuroscience, 2021, volume 12, pages 330-339; J. F. Quilez del Moral, C. R. Martínez, H. P. del Pulgar, J. E. M. Gonzelez, I. Fernendez, J. L. López-Pérez, A. Fernández-Arteaga and A. F. Barrero, “Synthesis of Cannabinoids: “In Water” and “On Water” Approaches: Influence of SDS Micelles”, The Journal of Organic Chemistry, 2021, volume 86, pages 3344-3355; J. Vacek, J. Vostalova, B. Papouskova, D. Skarupova, M. Kos, M. Kabelac and J. Storch, “Antioxidant function of phytocannabinoids: Molecular basis of their stability and cytoprotective properties under UV-irradiation”, Free Radical Biology and Medicine, 2021, volume 164, pages 258-270; D. Brumar, B. Geiling and M. Haghdoost Manjili, “Cannabichromene compositions and methods of synthesizing cannabichromene” Patent Application WO 2021/127786 A1; C. K. Marlow, “Preparation of cannabichromene and related cannabinoids”, Patent Application WO 2021/133989 A1; W.-C. Zhang and A. P. Honeycutt, “Methods of preparing synthetic cannabichromene and cannabicitran and derivatives thereof”, Patent Application WO 2021/222609). None of these reactions are suitable for large-scale manufacture of significant numbers of analogs with facile variation in the arene ring substituents provide teaching on the synthesis of single enantiomer cannabinoids.

Cannibichromenic acid (CBCA, 2) and related chromene acids have previously been synthesized on a small-scale via bromo-arene/lithium exchange and carboxylation or by alkali or amine-catalyzed cyclocondensation reactions of 5-alkyl-resorcylate esters with citral, via quinone-mediated oxidation reactions of substituted cyclohexanone-carboxylic esters, or via an oxidative cyclization reaction with 2,3-dichloro-5,6-dicyano-p-benzoquinone (R. Davis, J. Black, T. Smeltzer and S. Colvin, “Methods of synthesizing halogenated cannabinoids and derivatives of cannabinoids”, Patent Application WO 2022/192582; Y. R. Lee, J. H. Choi and S. H. Yoon, “Efficient and general method for the synthesis of benzopyrans by ethylenediamine diacetate-catalyzed reactions of resorcinols with α,β-unsaturated aldehydes. One step synthesis of biologically active (±)-confluentin and (±)-daurichromenic acid”, Tetrahedron Letters, 2005, volume 46, pages 7539-7543; M. Mondal, V. G. Puranik and N. P. Argade, “A Facile Phenol-Driven Intramolecular Diastereoselective Thermal/Base-Catalyzed Dipolar [2+2] Annulation Reactions: An Easy Access to Complex Bioactive Natural and Unnatural Benzopyran Congeners”, The Journal of Organic Chemistry, 2007, volume 72, pages 2068-2076; H. Hu, T. J. Harrison and P. D. Wilson, “A Modular and Concise Total Synthesis of (±)-Daurichromenic Acid and Analogues”, The Journal of Organic Chemistry, 2004, volume 69, pages 3782-3786; S. Morimoto, K. Komatsu, F. Taura and Y. Shoyama, “Enzymological Evidence for Cannabichromenic Acid Biosynthesis”, Journal of Natural Products, 1997, volume 60, pages 854-857). None of these reactions are suitable for large-scale manufacture of significant numbers of analogs with facile variation in the arene ring substituents nor provide teaching on the synthesis of single enantiomer cannabinoids.

There have been only limited studies of the synthesis and characterization of co-crystals derived from a cannabinoid and a co-crystal co-forming reagent. Co-crystal derivatives of racemic quinone-analogs of cannabichromene (CBC, 1) have been claimed. Co-crystallization of lower molecular weight cannabinoids namely cannabinol (CBN; m.p. 75-77° C.) and tetrahydrocannabinol (THC; m.p. 63-66° C.) with carnitine, aspartame, L-proline, D-proline, L-arginine, L-lysine, betaine, tetramethylpyrazine, 1H-imidazole, nicotinic acid, saccharin, urea, or nicotinamide are claimed to be useful to provide, presumably higher melting point solids, including CBN with tetramethylpyrazine or L-proline or D-proline being most extensively studied inter alia by powder X-ray diffraction analysis. Co-crystals of cannabinoids including THC, cannabidiol (CBD), or CBN with stilbenoids including resveratrol, piceatannolin, pinosylvin, astringin, piceid, oxyresveratrol, amelopsin A, amelopsin B, vitisin A, combretastatin, combretastatin B-1, isonotholaenic acid, combretastatin A-1, combretastatin A-4, gnetucleistol E, pinostilbene, pterostilbene, isoharpontigenin, gnetucleistol D, 4-methoxyresveratrol, rhaponticin, and rhapontigenin, cavicularin, 1-hydroxyphenanthrene or andjuncusol are claimed to be useful with carbohydrate adjuvants in oral dosage formulations (E. Muhoz Blanco and G. Appendino, “Chromenic phytocannabinoids, their synthesis and use in treatment or prevention of Disease”, EP 3,666,765 A1; E. Muhoz Blanco, G. Appendino and J. D. Uniciti-Broceta, “Chromenic phytocannabinoids, their synthesis and use in treatment or prevention of Disease”, WO 2020/120582 A1; G. Mkrtchyan, J. K. Hoerner, R. W. Couch, J. A. Bis and S. A. R. Carino, “Cocrystals of Cannabinoids”, WO 2021/138610 A1; G. Rehman and K. P. Bushfield, “Compositions comprising co-crystals of stilbenoids and cannabinoids”, Patent Application WO 2019/153088 A1; N. Tesson, C. Trilla and A. C. Comely, “Solid compositions of cocrystals of cannabinoids”, Patent Application WO 2020/089424 A1].

There have been no studies of the use of co-crystallization to resolve any racemic cannabinoid. Additionally, there have been no specific studies reported on the formation of any co-crystal derivative of cannabichromene (CBC, 1), cannabichromenic acid (CBCA, 2), cannabichromevarin (CBCV, 3), cannabichromevarinic acid (CBCVA, 4) or any structurally related non-quinone cannabinoid.

There have been but limited studies on the separated enantiomers of the chromene cannabinoids. It has been reported that naturally occurring cannabichromene (CBC, 1) may be resolved using chiral HPLC methods, however nothing has been reported on the resolution of other chromene cannabinoids or cannabinoid acids and nothing is known about the relative efficacy of the separated enantiomers against any biomedically relevant target or in cellular assays. The structurally related meroterpenoid glaudichaudianic acid (5), a prenylated chromene, isolated from Piper gaudichaudianu, has been resolved by HPLC and the (S)-enantiomer shown to be more efficacious as a potent trypanocidal compound against the Y-strain of Trypanosoma cruzi. The methanol extract of the Asian plant Rhododendron dauricum gave several anti-HIV-active chromenes, which are derived from the parent (+)-daurichromenic acid (6). This single enantiomer meroterpenoid chromene 6 has been synthesized via a multi-step sequence from farnesal.

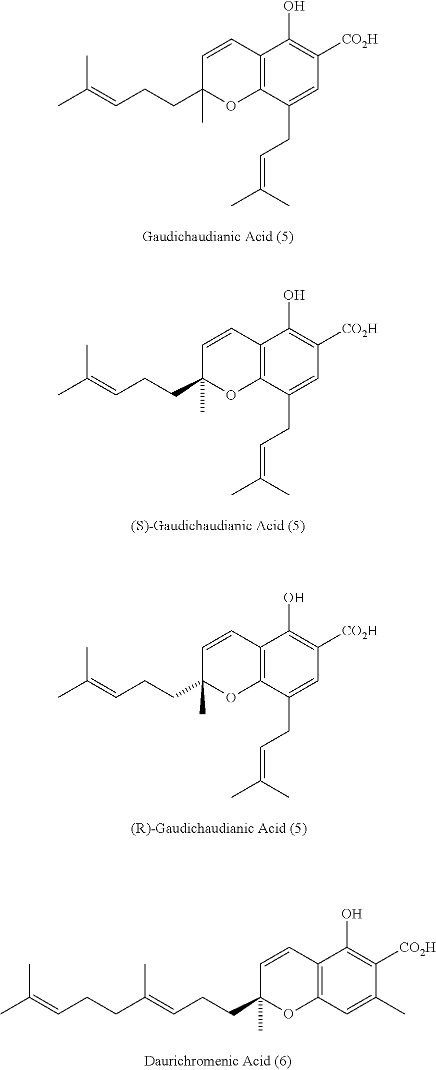 [SMILES: CC(C)=CCCC1(C)C=Cc2c(O)c(C(=O)O)cc(CC=C(C)C)c2O1 ; CC(C)=CCC[C@@]1(C)C=Cc2c(O)c(C(=O)O)cc(CC=C(C)C)c2O1 ; CC(C)=CCC[C@]1(C)C=Cc2c(O)c(C(=O)O)cc(CC=C(C)C)c2O1 ; CC(C)=CCC/C(C)=C/CC[C@@]1(C)C=Cc2c(cc(C)c(C(=O)O)c2O)O1]

For such resolutions see G. Mazzoccanti, O. H. Ismail, I. D'Acquarica, C. Villani, C. Manzo, M. Wilcox, A. Cavazzini and F. Gasparrini, “Cannabis through the looking glass: chemo- and enantio-selective separation of phytocannabinoids by enantioselective ultra-high performance supercritical fluid chromatography” Chemical Communications, 2017, volume 53, pages 12262 to 12265; J. M. Batista Jr., A. N. L. Batista, D. Rinaldo, W. Vilegas, D. L. Ambrósio, R. B. M. Cicarelli, V. S. Bolzani, M. J. Kato, L. A. Nafie, S. N. López and M. Furlan, M., “Absolute Configuration and Selective Trypanocidal Activity of Gaudichaudianic Acid Enantiomers”, Journal of Natural Products, 2011, volume 74, pages 1154 to 1160; A. Mándi, M. M. M. Swamy, T. Taniguchi, M. Anetai and K. Monde, “Reducing Molecular Flexibility by Cyclization for Elucidation of Absolute Configurations by CD Calculations: Daurochromenic Acid”, Chirality, 2016, volume 28, pages 453 to 459; M. M. M Swamy, A. Mándi, M. Anetai and K. Monde, “Stereochemistry of a Rhododaurochromanic Acid Derivative” Natural Product Communications, 2016, volume 11, pages 193 to 195; K. Liu and K.; Woggon, “Enantioselective Synthesis of Daurichromenic Acid and Confluentin”, European Journal of Organic Chemistry, 2010, pages 1033 to 1036; M. Okada, K. Saito, C. P. Wong, C. Li, D. Wang, M. Iijima, F. Taura, F. Kurosaki, T. Awakawa and I. Abe, “Combinatorial Biosynthesis of (+)-Daurichromenic Acid and Its Halogenated Analogue” Organic Letters, 2017, volume 19, pages 3183 to 3186; Z. Jin and Y. Kang, “Total Synthesis of Daurichromenic Acid” U.S. Pat. No. 7,102,020 B2; A. R. Agua, P. J. Barr, C. K. Marlowe and M. C. Pirrung, “Cannabichromene Racemization and Absolute Stereochemistry Based on a Cannabicyclol Analog”, The Journal of Organic Chemistry, 2021, volume 86, pages 8036-8040; J. M. Ferraro and W. J. Umstead, “Chiral Separation of Cannabichromene, Cannabicyclol, and Their Acidic Analogs on Polysaccharide Chiral Stationary Phases”, Molecules, 2023, volume 28, 1164 (16 pages); T. Onishi and W. J. Umstead, “The Separation of Cannabinoids on Sub-2 μm Immobilized Polysaccharide Chiral Stationary Phases”, Pharmaceuticals, 2021, volume 14, 1250 (11 pages); C. De Luca, A. Buratti, W. Umstead, P. Franco, A. Cavazzini, S. Felletti and M. Catani, “Investigation of retention behavior of natural cannabinoids on differently substituted polysaccharide-based chiral stationary phases under reversed-phase liquid chromatographic conditions”, Journal of Chromatography A, 2022, volume 1672, 463076 (8 pages); see also D. C. Metcalf, “Composition and methods to separate cannabinoids from impurities by crystallization”, Patent Application WO 2022/020743 A1.

Crystalline salts of cannabinoid carboxylic acids with achiral amines such as triethylamine, tributylamine, N,N-di-iso-propylethylamine, methyldicyclohexylamine, 1,4-diazabicyclo[2.2.2]octane, 1,5-diazabicyclo[4.3.0]non-5-ene, dicyclohexylamine, isopropylcyclohexylamine, and 2,2,6,6-tetramethylpiperidine have been synthesized but resolutions of racemic cannabinoid carboxylic acids with chiral amines have not been reported (T. Durst and J. van der Vlugt, “Methods for extraction, processing, and purification of minor cannabinoid compounds from cannabis”, Patent Application WO 2022/115971 A1).

Cannabichromene (CBC, 1) lacks significant affinity for CB₁ or CB₂ but it inhibits cellular uptake of anandamide. CBC (1) is the most potent phytocannabinoid agonist of the TRPA1 cation channels and also modulates other TRP channels with the activation of TRPV3 and TRPV4, the blocking of agonism of TRPV4 and the activation of TRPM8 but all at low potency. It has been reported to increase the viability of adult mouse neural stem/progenitor cells during differentiation and to show anti-inflammatory activities and antibacterial and antifungal properties. CBC (9) exerts anti-inflammatory actions in activated macrophages, with tonic CB1 cannabinoid signaling being negatively coupled to this effect, and ameliorates experimental murine colitis (S. E. Turner, C. M. Williams, L. Iversen and B. J. Whalley, “Molecular Pharmacology of Phytocannabinoids”, Phytocannabinoids, 2017, pages 61-101; A. Tubaro, A. Giangaspero, S. Sosa, R. Negri, G. Grassi, S. Casano, R. D. Loggia and G. Appendino, “Comparative topical anti-inflammatory activity of cannabinoids and cannabivarins”, Fitoterapia, 2010, volume 81, pages 816-819; C. E. Turner and M. A. Elsohly, “Biological activity of cannabichromene, its homologs and isomers”, Journal of Clinical Pharmacology, 1981, volume 21, Supplement pages 283-291; B. Romano, F. Borrelli, I. Fasolino, R. Capasso, F. Piscitelli, M. G. Cascio, R. G. Pertwee, D. Coppola, L. Vassallo, P. Orlando, V. Di Marzo and A. A. Izzo A, “The cannabinoid TRPA1 agonist cannabichromene inhibits nitric oxide production in macrophages and ameliorates murine colitis”, British Journal of Pharmacology, 2013, volume 169, pages 213-229).

There are limited studies on the biological effect of cannabichromevarin (CBCV, 3). It has been shown usually in combinations with other cannabinoid to have anti-inflammatory activities and has been claimed to be of use in both monotherapy and combination therapy for treating epilepsy. Additionally, CBCV (3) has been shown to be a modest inhibitor of homeodomain-interacting protein kinase 2, a conserved serine/threonine kinase, which regulates transcription, cell differentiation, proliferation and apoptosis. (Y. Shoyama, H. Hirano, M. Oda, T. Somehara and I. Nishioka, Cannabis IX Cannabichromevarin and cannabigerovarin, two new propyl homologs of cannabichromene and cannabigerol”, Chemical & Pharmaceutical Bulletin, 1975, volume 23, pages 1894-1895; A. Tubaro, A. Giangaspero, S. Sosa, R. Negri, G. Grassi, S. Casano, R. D. Loggia and G. Appendino, “Comparative topical anti-inflammatory activity of cannabinoids and cannabivarins”, Fitoterapia, 2010, volume 81, pages 816-819; V. Stahl, “Cannabis and Derivatives Thereof for the Treatment of Pain and Inflammation Related with Dental Pulp and Bone Regeneration Related to Dental Jaw Bone Defects”, WO Patent Application 2019/030762 A2; G. Wang, L. Zhu, Y. Zhao, S. Gao, D. Sun, J. Yuan, Y. Huang, X. Zhang and X. Yao, “A natural product from Cannabis sativa subsp. sativa inhibits homeodomain-interacting protein kinase 2 (HIPK2), attenuating MPP+-induced apoptosis in human neuroblastoma SH-SY5Y cells”, Bioorganic Chemistry, 2017, volume 72, pages 64-73; B. Whalley, C. Williams, G. Stephens and T. Futamura, “Use of the Phytocannabinoid Cannabidivarin (CBDV) in the Treatment of Epilepsy”, U.S. Pat. No. 9,125,859).

The cannabinoid carboxylic acids cannabichromenic acid (CBCA, 2) and cannabichromevarinic acid (CBCVA, 4) currently have limited biological and medical applications. Other cannabinoid carboxylic acids have reported biological properties. Cannabigerolic acid (CBGA) has been claimed to be a modest modulator of the inhibition of ovarian, breast, lung, pancreas and other cancer cell growth by cannabidiol (CBD) and cannabigerol (CBG) and itself to kill breast cancer cells. It is an inverse agonist of the G-protein Coupled Receptor GPR55, an antagonist of mono-acyl-glyceride lipase and a dual PPARα/γ agonist. Cannabigerolic acid (CBGA) has also been suggested to show analgesic effects. Cannabigerovarinic acid (CBGVA) is reported to have anticancer cytostatic effects at high doses in vitro on leukemia cells. It has been claimed that mixtures of CBCA (2), CBCVA (4) or another cannabinoid with mitragynine, pseudoindoxyl or 7-hydroxymitragynine and another additive may be used to treat inflammation, spasms or pain. Based alone on an in vitro cellular assay, cannabichromenic acid (CBCA, 2), amongst other acidic cannabinoids, have been claimed to be of use in increasing the natural resistance of an animal, enhancing cellular resistance, for treating diabetes or atherosclerosis and in reducing the decline in stress response found in ageing. Formulations comprising of cannabichromenic acid (CBCA, 2) or another cannabinoid with an isotonic agent, a surfactant and one or more stability enhancers have been claimed as neuroprotective agents and anti-convulsive agents, particularly in the treatment of newborn hypoxic-ischemic encephalopathy, status epilepticus or stroke. (E. D'Aniello, T. Fellous, F. A. lannotti, A. Gentile, M. Allara, F. Balestrieri, R. Gray, P. Amodeo, R. M. Vitale and V. Di Marzo, “Identification and characterization of phytocannabinoids as novel dual PPARα/γ agonists by a computational and in vitro experimental approach”, Biochimica et Biophysica Acta General Subjects, 2019, volume 183, pages 586-597; H. A. A. J. Korthout, K. C. M. Verhoeckx, R. F. Witkamp, R. P. Doornbos and M. Wang, “Medicinal Acidic Cannabinoids, U.S. Pat. No. 7,807,711; D. Parolaro, P. Massi, A. Antonio, F. Borelli, G. Aviello, V. Di Marzo, L. De Petrocellis, A. S. Schiano Moriello, A. Ligresti, A.; R. A. Ross, L. A. Ford, S. Anavi-Goffer, M. Guzman, G. Velasco, M. Lorente, S. Torres, T. Kikuchi, G. Guy, C. Stott, S. Wright, A. Sutton, D. Potter and E. De Meijer, “Phytocannabinoids in the Treatment of Cancer”, U.S. Pat. No. 8,790,719; F. A. Javid, M. Duncan and C. Stott, “Use of Phytocannabinoids in the Treatment of Ovarian Carcinoma”, U.S. Pat. No. 10,098,867; C. Stott, M. Duncan and T. Hill, “Active Pharmaceutical Ingredient (API) Comprising Cannabinoids for use in the Treatment of Cancer”, U.S. Pat. No. 9,962,341; K. A. Scott, S. Shah, A. G. Dalgleish and W. M. Liu, “Enhancing the Activity of Cannabidiol and Other Cannabinoids In Vitro Through Modifications to Drug Combinations and Treatment Schedules”, Anticancer Research, 2013, volume 33, pages 4373-4380; S. A. Ahmed, S. A. Ross, D. Slade, M. M. Radwan, F. Zulfiqar and M. A. EISohly, “Cannabinoid Ester Constituents from High-Potency Cannabis sativa”, Journal of Natural Products, 2008 volume 71, pages 536-542; Kariman, A., “Compound and Method for Treating Spasms, Inflammation and Pain”, US Patent Application US 2018/0193399 A1; H. A. A. J. Korthout, “Medical use for Acidic Cannabinoids”, WO Patent Application 2012/144892 A1; S. Wright and J. Wilhu, Parenteral formulations”, GB Application 2551986).

A vast number of combinations of one, two or three cannabinoids including cannabichromenic acid (CBCA, 2) or cannabichromevarinic acid (CBCVA, 4) admixed with terpenes have been claimed but their possible uses have not been defined (K. Levy, J. M. Cooper, J. R. Martin and B. G. Reid, “Compositions Purposefully Selected Comprising Purified Cannabinoids and/or Purified Terpenes”, WO Patent Application 2018/160827 A1).

In contrast to these currently limited biomedical applications for the cannabinoid acids 2 and 4, THCA, which is the carboxylic acid precursor of THC, has been widely studied. In a series of preclinical studies, THCA has been shown to be of value in controlling pain including neuropathic pain and fibromyalgia, epilepsy, cancers of the prostate, breast, colon, lung and skin, inflammation including encephalomyelitis as well as autoimmune diseases and to act as an anti-emetic (for examples see R. Z. Dejana, M. Folid, Z. Tantoush, M. Radovanovid, G. Babid and S. M. Janković, “Investigational cannabinoids in seizure disorders, what have we learned thus far?” Expert Opinion on Investigational Drugs, 2018, volume 27, pages 535-541; E. M. Rock, R. L. Kopstick, C. L. Limebeer and L. A. Parker, “Tetrahydrocannabinolic acid reduces nausea-induced conditioned gaping in rats and vomiting in Suncus murinus”, British Journal of Pharmacology, 2013, volume 170, pages 641-648; H. A. A. J. Korthout, K. C. M. Verhoeckx, R. F. Witkamp, R. P. Doornbos and M. Wang, “Medicinal Acidic Cannabinoids”, U.S. Pat. No. 7,807,711 B2; E. M. Rock, C. L. Limebeer, R. Navaratnam, M. A. Sticht, N. Bonner, K. Engeland, R. Downey, H. Morris, M. Jackson and L. A. Parker, “A comparison of cannabidiolic acid with other treatments for anticipatory nausea using a rat model of contextually elicited conditioned gaping”, Psychopharmacology, 2014, volume 231, pages 3207-3215; V. Di Marzo, L. De Petrocellis and A. S. Moriello, “New use for cannabinoid-containing plant extracts”, G. B. Patent 2,448,535; D. Parolaro, P. Massi, A. Antonio, F. Borelli, G. Aviello, V. Di Marzo, L. De Petrocellis, A. S. Schiano Moriello, A. Ligresti, A.; R. A. Ross, L. A. Ford, S. Anavi-Goffer, M. Guzman, G. Velasco, M. Lorente, S. Torres, T. Kikuchi, G. Guy, C. Stott, S. Wright, A. Sutton, D. Potter and E. De Meijer, “Phytocannabinoids in the Treatment of Cancer”, U.S. Pat. No. 8,790,719; Trevor Percival Castor, T. P.; Rosenberry, L. C.; Tyler, T. A.; Student, R. J., “Methods for Making Compositions and Compositions for Treating Pain and Cachexia”, US Patent Application 2008/0103193 A1; K. Kariman, “Compound and Method for Treating Spasms, Inflammation and Pain”, US Patent Application 2018/0193399 A1; A. Sinai and Z. Turner, “Use of Cannabis to Treat Fibromyalgia, Methods and Compositions Thereof”, WO Patent Application 2016/181394 A1).

If the cannabinoid acids 2 and 4 were to be made available more easily in larger quantities and higher purities, it would be possible to better and more thoroughly examine their uses in medicine either as mono-therapeutic agents or in combination with other cannabinoids or other biologically active compounds. It is germane to note that mixtures of cannabinoids may be more efficacious than single components (the entourage effect). For example, the presence of THCA and other cannabinoids has been shown to enhance the efficacy of THC as an antitumor agent in cell culture and animal models of ER+/PR+, HER2+ and triple-negative breast cancer (for example see S. Blasco-Benito, M. Seijo-Vila, M. Caro-Villalobosa, I. Tundidor, C. Andradas, E. García-Taboada, J. Wade, S. Smith, M. Guzmán, E. Pérez-Gómez, M. Gordon and C. Sánchez, “Appraising the “entourage effect”: Antitumor action of a pure cannabinoid versus a botanical drug preparation in preclinical models of breast cancer”, Biochemical Pharmacology, 2018, volume 157, pages 285-293).

The present invention is directed towards overcoming the problems of availability of all the cannabinoids 1 to 4 in high purities by providing efficient/reproducible manufacturing routes for these compounds and providing flexible syntheses of novel cannabinoid analogs, which may be used as active compounds either alone or admixed in combination with known cannabinoids or other drugs in drug formulations for the treatment of infections, pain, multiple sclerosis-related spasticity, nausea, anorexia, epilepsy, Alzheimer's and neurodegenerative diseases, brain injury/concussion/traumatic brain injury, stroke, cancer, reduction of inflammation and immuno-inflammation related diseases, diseases/injury of the eye including but not limited to glaucoma, dry eye, corneal injury or disease and retinal degeneration or disease, disorders of immune-inflammation, lung injury or disease, liver injury or disease, kidney injury or disease, pancreatitis and disorders of the pancreas cardiovascular injury or disease, and organ transplant, reduction of post-surgical inflammation, treatment or prevention of skin diseases among other diseases, and as anti-oxidants.

### SUMMARY OF THE INVENTION

Among the benefits and improvements disclosed herein, other objects and advantages of the disclosed embodiments will become apparent from the following wherein like numerals represent like parts throughout the several figures. Detailed embodiments of cannabinoid compounds, intermediary compounds, and a process for preparation of cannabinoid and cannabimimetic compounds and their intermediaries are disclosed; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “In some embodiments” and “in some embodiments” as used herein do not necessarily refer to the same embodiment(s), though it may. The phrase “in another embodiment” as used herein does not necessarily refer to a different embodiment, although it may. Thus, as described below, various embodiments may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.

Further, the terms “substantial,” “substantially,” “similar,” “similarly,” “analogous,” “analogously,” “approximate,” “approximately,” and any combination thereof mean that differences between compared features or characteristics is less than 25% of the respective values/magnitudes in which the compared features or characteristics are measured and/or defined.

The purpose of combination or adjuvant therapies herein described are to enhance the efficacy of a drug by the use of a second drug or more drugs or to reduce the dose-limiting toxicities of a drug by the use of a second drug or more drugs.

As used herein, the term “optionally substituted benzyl” means a benzyl group optionally bearing 1, 2 or 3 independently varied C1-C4 alkyl, C1-C4 alkyloxy, fluoro, chloro, hydroxy, trifluoromethyl, trifluoromethoxy, methylenedioxy, cyano, or methoxymethyl groups at an aromatic ring position or positions or 1 or 2 independently varied C1-C4 alkyl at the benzylic methylene.

As used herein, the term “optionally substituted 2-phenylethyl” means a 2-phenylethyl group optionally bearing 1, 2 or 3 independently varied C1-C4 alkyl, C1-C4 alkyloxy, fluoro, chloro, hydroxy, trifluoromethyl, trifluoromethoxy, methylenedioxy, cyano, or methoxymethyl groups at an aromatic ring position or positions or 1 or 2 independently varied C1-C4 alkyl at either or both methylene groups.

If not otherwise defined herein, the term “optionally substituted aryl” means a phenyl ring optionally bearing 1, 2, or 3 independently varied C1-C4 alkyl, C1-C4 alkyloxy, fluoro, or chloro groups.

If not otherwise defined herein, the term “substituted” means optionally substituted at any position with varied C1-C4 alkyl, C1-C4 alkyloxy, fluoro, chloro, hydroxy, trifluoromethyl, trifluoromethoxy, methylenedioxy, cyano, or methoxymethyl groups.

The present invention relates to a process for the preparation of diverse known and novel racemic cannabinoids 7 from the precursors 8 via the racemic intermediate 9 including racemic cannabichromene (CBC, 1), racemic cannabichromenic acid (CBCA, 2), racemic cannabichromevarin (CBCV, 3) and racemic cannabichromevarinic acid (CBCVA, 4) and other naturally occurring racemic bicyclic cannabinoids and other synthetic racemic bicyclic analogues from simple inexpensive starting materials using a cascade sequence of allylic rearrangement and aromatization to prepare dioxinones 8 and subsequent oxidative cyclization to produce dioxinones 9 and conversion into the racemic cannabinoids 7. The present invention also relates to processes for the resolution of racemic cannabichromene (CBC, 1), racemic cannabichromevarin (CBCV, 3), racemic cannabichromenic acid (CBCA, 2), racemic cannabichromevarinic acid (CBCVA, 4) and other racemic bicyclic neutral cannabinoids and cannabinoid carboxylic acids by direct separation of the enantiomers by HPLC, or by formation of diastereoisomeric co-crystals with a chiral co-crystallization agent (CCA) and their separation and reconversion to the resolved cannabinoid, or by formation of diastereoisomeric salts from racemic cannabichromenic acid (CBCA, 2), racemic cannabichromevarinic acid (CBCVA, 4) and related racemic cannabinoid acids with chiral amines (CA) and the conversion of these salts with alkali or base and subsequent acidification with optional decarboxylation into single enantiomer cannabichromene (CBC, 1), single enantiomer cannabichromenic acid (CBCA, 2), single enantiomer cannabichromevarin (CBCV, 3), single enantiomer cannabichromevarinic acid (CBCVA, 4) and analogous single enantiomer cannabinoids of both enantiomeric series as well as scalemic mixtures of such pairs of enantiomers.

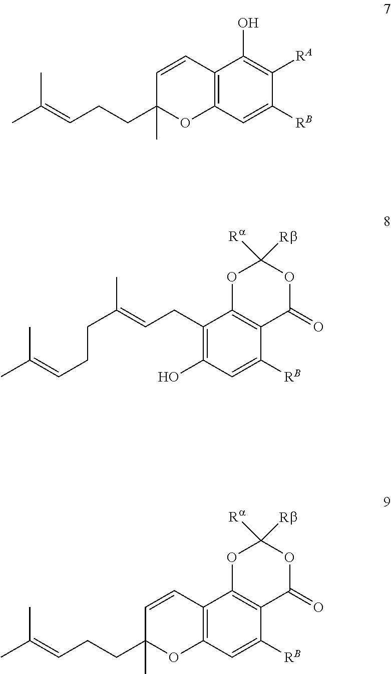

wherein:
   R^{A} is H, CO₂H and its pharmaceutically acceptable salts or co-crystals, CO₂R^{C}, CONHR^{D}, CONR^{D}R^{E};
   R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{C} is C₁ to C₆ alkyl, (CH₂)_{q}—C₃ to C₆ cycloalkyl, allyl, optionally substituted benzyl or optionally substituted 2-phenylethyl;
   q is 0, 1, 2, 3, 4, 5 or 6;
   R^{D} is C₁ to C₆ alkyl, (CH₂)ᵣ—C₃ to C₆ cycloalkyl, allyl, optionally substituted benzyl or optionally substituted 2-phenylethyl; R^{E} is C₁ to C₆ alkyl, (CH₂)ᵣ—C₃ to C₆ cycloalkyl, allyl, optionally substituted benzyl or optionally substituted 2-phenylethyl; or NR^{D}R^{E} is azetidinyl, pyrrolidinyl, morpholinyl or piperidinyl each optionally substituted by one or two hydroxyl groups or one hydroxymethyl group with the exception that the hydroxyl groups cannot be on the carbon bearing the heterocyclic ring nitrogen or the heterocyclic ring oxygen with morpholine;
   R^{F} is C₁ to C₆ alkyl, (CH₂)ᵣ—C₃ to C₆ cycloalkyl;
   each r is independently 0, 1, 2, 3, 4, 5 or 6;
   Rα is C₁ to C₆ alkyl or optionally substituted aryl, and preferably methyl; and
   Rβ is C₁ to C₆ alkyl or optionally substituted aryl, and preferably methyl; or
   Rα and Rβ in combination are (CH₂)ₛ (s is 4, 5 or 6);
   any optional hydroxyl groups in the R^{B} substituent in structures 8 and 9 is protected.

The synthetic methods are suitable for use on a large scale and for manufacturing purposes of racemic mixtures, single enantiomers or scalemic mixtures. Examples of known cannabinoids that are available using the synthetic routes are cannabichromene (CBC, 1), cannabichromenic acid (CBCA, 2), cannabichromevarin (CBCV, 3), and cannabichromevarinic acid (CBCVA, 4).

The synthetic methods are also suitable for the synthesis of racemic mixtures, single enantiomers or scalemic mixtures of the novel cannabinoids 7 and such novel compounds are also part of the invention. The racemic cannabinoids 7 below, which are novel analogs of cannabichromene (CBC, 1), cannabichromenic acid (CBCA, 2), cannabichromevarin (CBCV, 3) and cannabichromevarinic acid (CBCVA, 4), are also available by the synthetic routes herein described and are also part of the invention.

The synthetic methods are suitable for the synthesis of known and novel cannabinoids 7 as single enantiomers of the (S) or (R) enantiomeric series by direct separation of the enantiomers by HPLC, or by formation of diastereoisomeric co-crystals and their separation and reconversion to the resolved cannabinoid, or by formation of diastereoisomeric salts, esters or amides from cannabinoid carboxylic acids (7, R^{A}═CO₂H) with chiral amines or chiral alcohols and the conversion of the separated salts, esters or amides with alkali or base and subsequent acidification with optional decarboxylation into single enantiomer cannabinoid carboxylic acids (7, R^{A}═CO₂H) or neutral cannabinoids (7, R^{A}═H) as well as analogous cannabinoids of both enantiomeric series. Such resolutions may be used to produce non-racemic scalemic mixtures of cannabinoids (S)-7 and (R)-7 as well as single enantiomers.

These novel single enantiomer cannabinoids (S)-7 and (R)-7 include the single enantiomers of cannabichromenic acid (CBCA, 2) and cannabichromevarinic acid (CBCVA, 4) as well as all their analogs and all are also part of the invention.

These novel single enantiomer cannabinoids (S)-7 and (R)-7 have the formulae:

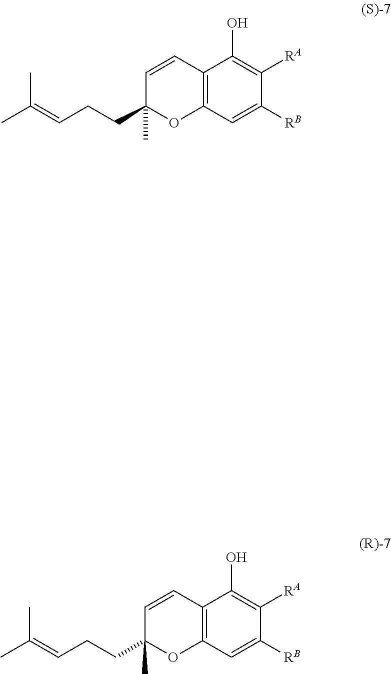

wherein:
   R^{A} is H;
   R^{B} is H, methyl, ethyl, n-butyl, n-hexyl, n-heptyl, n-octyl, n-nonyl or n-decyl in each case optionally substituted by one or two hydroxyl groups or optionally substituted by one or more fluoro-groups; or n-propyl or n-hexyl substituted by one or two hydroxyl groups or substituted by one or more fluoro-groups; or branched C₃ to C₁₀ alkyl or double branched C₄ to C₁₀ alkyl in each case optionally substituted by one or two hydroxyl groups or optionally substituted by one or more fluoro-groups; or (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{F} is C₁ to C₆ alkyl, (CH₂)ᵣ—C₃ to C₆ cycloalkyl;
   each r is independently 0, 1, 2, 3, 4, 5 or 6;
   or
   R^{A} is CO₂H and its pharmaceutically acceptable salts or co-crystals, CO₂R^{C}, CONHR^{D}, CONR^{D}R^{E};
   R^{B} is H, methyl, ethyl, n-propyl, n-butyl, n-pentyl, n-hexyl, n-heptyl, n-octyl, n-nonyl or n-decyl in each case optionally substituted by one or two hydroxyl groups or optionally substituted by one or more fluoro-groups; or branched C₃ to C₁₀ alkyl or double branched C₄ to C₁₀ alkyl in each case optionally substituted by one or two hydroxyl groups or optionally substituted by one or more fluoro-groups; or (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{C} is C₁ to C₆ alkyl, (CH₂)_{q}—C₃ to C₆ cycloalkyl, allyl, optionally substituted benzyl or optionally substituted 2-phenylethyl;
   q is 0, 1, 2, 3, 4, 5 or 6;
   R^{D} is C₁ to C₆ alkyl, (CH₂)ᵣ—C₃ to C₆ cycloalkyl, C₃ to C₆ cycloalkyl, allyl, optionally substituted benzyl or optionally substituted 2-phenylethyl; R^{E} is C, to C₆ alkyl, (CH₂)ᵣ—C₃ to C₆ cycloalkyl, allyl, optionally substituted benzyl or optionally substituted 2-phenylethyl; or NR^{D}R^{E} is azetidinyl, pyrrolidinyl, morpholinyl or piperidinyl each optionally substituted by one or two hydroxyl groups or one hydroxymethyl group with the exception that the hydroxyl groups cannot be on the carbon bearing the heterocyclic ring nitrogen or the heterocyclic ring oxygen with morpholine;
   R^{F} is C₁ to C₆ alkyl, (CH₂)ᵣ—C₃ to C₆ cycloalkyl;
   each r is independently 0, 1, 2, 3, 4, 5 or 6;
with the exception that each hydroxyl group in R^{B} cannot be on the carbon bearing a fluoro group or a second hydroxyl group.

The aforementioned novel cannabinoids with the limited formulae 7 as racemic mixtures, single enantiomers (S)-7 or (R)-7 or scalemic mixtures above may be used as active compounds either alone or admixed in combination with known cannabinoids such as but not limited to Δ⁹-tetrahydrocannabinol (THC), tetrahydrocannabivarin (THCV), cannabidiol (CBD) or cannabidivarin (CBVD) alone or in combination or with other drugs for the treatment of infections, pain, multiple sclerosis-related spasticity, nausea, epilepsy, Alzheimer's brain injury/concussion, cancer, glaucoma and retinal degeneration, disorders of immune-inflammation, lung injury or disease, liver injury or disease, kidney injury or disease, eye injury or disease, amongst other pathologies. In some embodiments, the said novel cannabinoids with the limited formulae 7 above either alone or admixed in combination with known cannabinoids such as but not limited to Δ⁹-tetrahydrocannabinol (THC), tetrahydrocannabivarin (THCV), cannabidiol (CBD), or cannabidivarin (CBDV) alone or in combination or with other drugs are formulated into pharmaceutical compositions in a suitable form for administration to a patient. Such formulations, in addition to the active cannabinoid or cannabinoids or other drugs in a combination therapeutic agent, contain pharmaceutically acceptable diluents and excipients. In the context of this invention, the term excipient encompasses standard excipients well known to a person of ordinary skill in the art (for example see Niazi, S. K., “Handbook of Pharmaceutical Manufacturing Formulations, Compressed Solid Products, 2009, volume 1, pages 67 and 99-169 2^{nd} Edition, Informa Healthcare) but also may include a volatile or mixture of volatile synthetic or isolated monoterpenes from Cannabis sativa and citrus oil. The aforementioned pharmaceutical compositions may be administrated to a patient by enteral, sublingual, intranasal, inhalation, rectal or parenteral drug administration or by other known methods of clinical administration.

### DETAILED DESCRIPTION OF THE INVENTION

### Large Scale-Synthesis and Resolution of Cannabichromene (CBC, 1), Cannabichromenic Acid (CBCA, 2), Cannabichromevarin (CBCV, 3), Cannabichromevarinic Acid (CBCVA, 4) and Analogs

The present invention relates to a process for the large-scale preparation of diverse known and novel cannabinoids 7 including cannabichromene (CBC, 1), cannabichromenic acid (CBCA, 2), cannabichromevarin (CBCV, 3), cannabichromevarinic acid (CBCVA, 4) and other naturally occurring bicyclic cannabinoids from simple inexpensive starting materials using a cascade sequence of oxidative cyclization and dioxinone ring opening. The invention includes synthesis of the target cannabinoids as oils or crystalline derivatives, as appropriate, including solvates, hydrates and polymorphs. The process involves the large-scale syntheses of racemic cannabinoids 7 and their resolution into the single enantiomer cannabinoids (S)-7 and (R)-7:

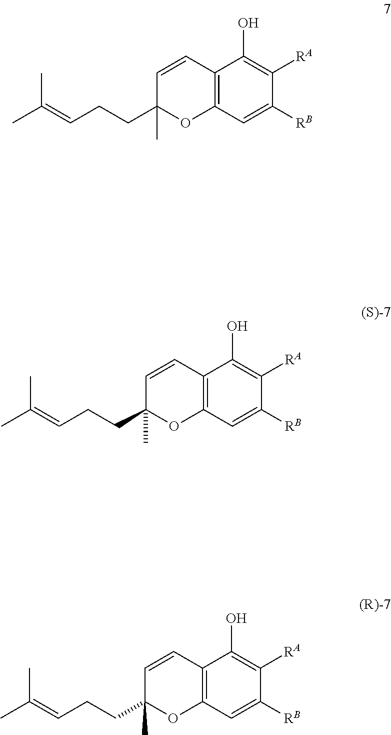

wherein:
   R^{A} is H, CO₂H and its pharmaceutically acceptable salts or co-crystals, CO₂R^{C}, CONHR^{D}, CONR^{D}R^{E}.
   R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{C} is C₁ to C₆ alkyl, (CH₂)_{q}—C₃ to C₆ cycloalkyl, allyl, optionally substituted benzyl or optionally substituted 2-phenylethyl;
   q is 0, 1, 2, 3, 4, 5 or 6;
   R^{D} is C₁ to C₆ alkyl, (CH₂)ᵣ—C₃ to C₆ cycloalkyl, allyl, optionally substituted benzyl or optionally substituted 2-phenylethyl; R^{E} is C₁ to C₆ alkyl, (CH₂)ᵣ—C₃ to C₆ cycloalkyl, allyl, optionally substituted benzyl or optionally substituted 2-phenylethyl; or NR^{D}R^{E} is azetidinyl, pyrrolidinyl, morpholinyl or piperidinyl each optionally substituted by one or two hydroxyl groups or one hydroxymethyl group with the exception that the hydroxyl groups cannot be on the carbon bearing the heterocyclic ring nitrogen or the heterocyclic ring oxygen with morpholine;
   R^{F} is C₁ to C₆ alkyl, (CH₂)ᵣ—C₃ to C₆ cycloalkyl;
   each r is independently 0, 1, 2, 3, 4, 5 or 6;
   said process comprising:
   treating a first intermediate of the formula 8 with a stoichiometric oxidant, an optional second oxidant, an optional additive or optional additives and a palladium catalyst in a solvent at 40 to 100° C. to provide the racemic intermediate 9 and secondly hydrolysis of said racemic dioxinone 9 with an alkali or base in a protic medium and acidification to provide the racemic carboxylic acids 7 [R^{A}═CO₂H] or by such hydrolysis and decarboxylation the racemic neutral cannabinoid 7 [R^{A}═H], or by such hydrolysis and esterification the racemic cannabinoid esters 7 [CO₂R^{C}], or by such hydrolysis and amide formation the racemic cannabinoid amides 7 [CONHR^{D}, CONR^{D}R^{E}];

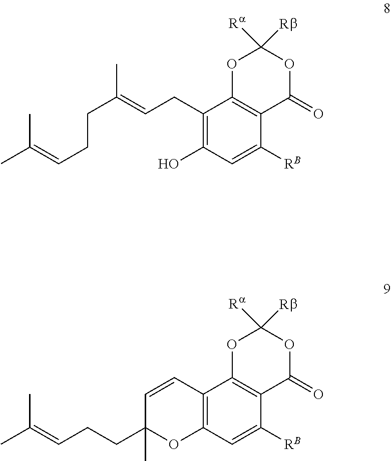

wherein:
   R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{F} is C₁ to C₆ alkyl, (CH₂)ᵣ—C₃ to C₆ cycloalkyl;
   r is independently 0, 1, 2, 3, 4, 5 or 6;
   Rα and Rβ are independently C₁ to C₆ alkyl or optionally substituted aryl or Rα and Rβ in combination are (CH₂)ₛ (s is 4, 5 or 6) with Rα and Rβ being preferably both methyl;
   any optional hydroxyl groups in the R^{B} substituent in structures 8 and 9 are protected;
   and separating the racemic carboxylic acids 7 [R^{A}═CO₂H], racemic neutral cannabinoid 7 [R^{A}═H], racemic cannabinoid esters 7 [CO₂R^{C}], or racemic cannabinoid amides 7 [CONHR^{D}, CONR^{D}R^{E}] by chromatography on a chiral phase such as by chiral-HPLC to provide the corresponding single enantiomer cannabinoids (S)-7 and (R)-7;
or
   separating the diastereoisomeric cannabinoid esters 7 [R^{A}═CO₂R^{G}], or cannabinoid amides 7 [R^{A}═CONR^{H}R^{I}] derived from the reaction of the racemic cannabinoid acids 7 [R^{A}═CO₂H] with single enantiomer chiral alcohols [R^{G}OH], or single enantiomer chiral amines [R^{H}R^{I}NH] by chromatography on a normal phase such as by column chromatography or HPLC and hydrolysis with optional decarboxylation to provide the corresponding single enantiomer cannabinoids (S)-7 and (R)-7 (R^{A}═CO₂H or H);
or
   treating the racemic carboxylic acids 7 [R^{A}═CO₂H], racemic neutral cannabinoid 7 [R^{A}═H], racemic cannabinoid esters 7 [CO₂R^{C}], or racemic cannabinoid amides 7 [CONHR^{D}, CONR^{D}R^{E}] with a chiral co-crystallization reagent (CCR), separating of the two diastereoisomeric co-crystals and reconverting each into the corresponding single enantiomer cannabinoids (S)-7 and (R)-7;
or
   treating the racemic carboxylic acids 7 [R^{A}═CO₂H] with chiral amines (CA), separation the two diastereoisomeric salts 10 and 11 and converting these salts 10 and 11 with alkali or base into the resolved cannabinoid acids (S)-7 and (R)-7 [R^{A}═CO₂H], or by such hydrolysis with subsequent decarboxylation into the resolved cannabinoids (S)-7 and (R)-7 [R^{A}═H], or by such hydrolysis and esterification into the resolved cannabinoid esters (S)-7 and (R)-7 [CO₂R^{C}], or by such hydrolysis and amide formation into the resolved cannabinoid amides (S)-7 and (R)-7 [CONHR^{D}, CONR^{D}R^{E}];
wherein
   R^{G}—OH is a single enantiomer chiral alcohol such as but not limited to menthol, 2-octanol, α-methylfenchol, methyl mandelate, or 1-phenylethanol;
   R^{H}R^{I}NH is a single enantiomer chiral amine such as but not limited to α-methylbenzylamine, α-methylnaphthylamine, leucine methyl ester, 1-amino-2-propanol, dimethyl aspartate, ephedrine, dimethyl glutamate, or phenylalanine methyl ester.

In general, separation of the two diastereoisomeric salts 10 and 11 by fractional recrystallization or other techniques known to a person of ordinary skill in the art as taught in standard textbooks [for example see Jaques, J.; Collet, A.; Wilen, S. H., “Enantiomers, Racemates and Resolutions”, John Wiley & Sons, New York, 1981, section 5.1.1, “Resolution of Acids” pages 257 to 259, 283 to 295, 299 to 328, and 329]; or in condensing said racemic carboxylic acids 7 [R^{A}═CO₂H] with a chiral alcohol (R^{G}—OH) or chiral amine (R^{H}R^{I}NH) to produce the corresponding mixture of diastereoisomeric esters 12 and 13 or diastereoisomeric amides 14 and 15, separation of the two esters or amides by fractional recrystallization or chromatography or by other techniques known to a person of ordinary skill in the art as taught in Jaques, Collet, and Wilen or of common general knowledge; and secondly hydrolysis of said esters 12 or 13 or amides 14 or 15 with optional deprotection as appropriate to provide the single enantiomer carboxylic acids (S)-7 and (R)-7 [R^{A}═CO₂H]; optionally decarboxylating the single enantiomer carboxylic acids (S)-7 and (R)-7 [R^{A}═CO₂H] to provide the neutral cannabinoids (S)-7 and (R)-7 [R^{A}═H] or converting said carboxylic acids (S)-7 and (R)-7 [R^{A}═CO₂H] by esterification or by amide formation with optional deprotection of protected hydroxyl groups in the R^{B} substituent, if present, as appropriate to provide single enantiomer cannabinoid esters and amides (S)-7 and (R)-7:

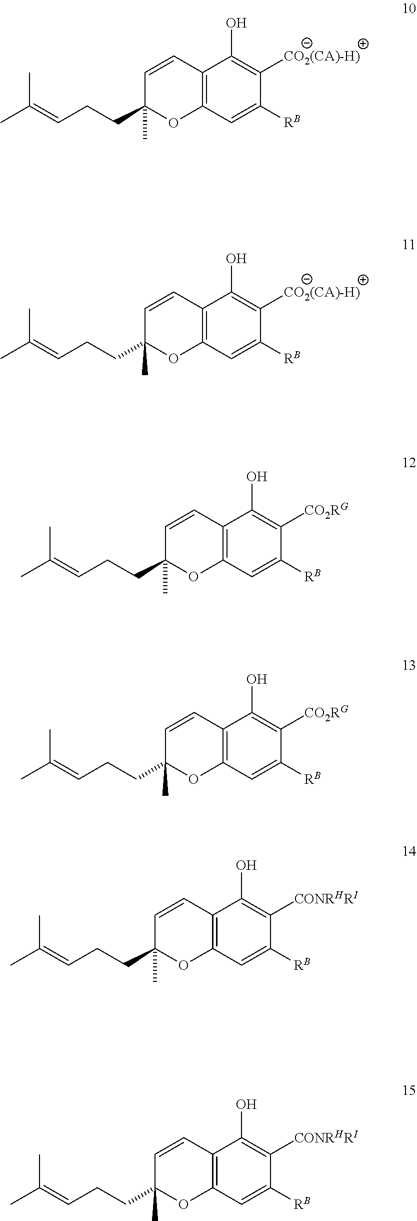

wherein:
   the stoichiometric oxidant is air, oxygen, a quinone, preferably p-benzoquinone, hydrogen peroxide or an alkyl peroxide, preferably t-butyl hydroperoxide;
   the optional second oxidant is a quinone, preferably p-benzoquinone, which is used with air or oxygen as the stoichiometric oxidant;
   the optional additive is an alkali metal hydrogencarbonate salt, preferably sodium hydrogencarbonate or potassium hydrogencarbonate;
   the optional additives are an alkali metal hydrogencarbonate salt, preferably sodium hydrogencarbonate or potassium hydrogencarbonate, and a copper(II) carboxylate, preferably copper(II) acetate;
   the palladium catalyst is a palladium(II) carboxylate, preferably palladium(II) acetate;
wherein:
   the optional hydroxyl-protecting group in R^{B} or groups are silyl protecting groups;
   the optional hydroxyl-protecting group or groups are preferably independently t-butyldimethylsilyl, thexyldimethylsilyl, t-butyldiphenylsilyl or tri-iso-propylsilyl protecting groups.

In the preferred embodiment, the oxidative cyclization is carried out with the optional second catalytic oxidant is p-benzoquinone, which is used with air or oxygen as the stoichiometric oxidant and with sodium hydrogencarbonate and copper(II) acetate as additives in DMSO and water as solvents at 60° C.; CA is a chiral amine such as but not limited to quinine, cinchonidine, quinidine, cinchonine, quinicine, cinchonicine, ephedrine, α-methylbenzylamine, α-methylnaphthylamine, leucine methyl ester, or tyrosine hydrazide;
R^{G}—OH is a chiral alcohol such as but not limited to menthol, 2-octanol, α-methylfenchol, methyl mandelate, or 1-phenylethanol;
R^{H}R^{I}NH is a chiral amine such as but not limited to α-methylbenzylamine, α-methylnaphthylamine, leucine methyl ester, 1-amino-2-propanol, dimethyl aspartate, ephedrine, dimethyl glutamate, phenylalanine methyl ester.

The small-scale syntheses of intermediates 8 and 9 (Rα and Rβ are both methyl; R^{B} is Me) are known [D. C. Elliott, T. K. Ma, A. Selmani, R. Cookson, P. J. Parsons and A. G. M. Barrett, Sequential Ketene Generation from Dioxane-4,6-dione-Keto-Dioxinones for the Synthesis of Terpenoid Resorcylates, Organic Letters 2016, 18, 1800-1803 and references therein]. However, methods for the efficient cost-effective large-scale synthesis of the novel cannabinoids 7 listed above have not been hitherto published nor is there any prior art on the resolutions and composition of matter of the single enantiomers of the racemic cannabinoids 7 including the single (S)- and (R)-enantiomers of cannabichromenic acid (CBCA, 2), cannabichromevarinic acid (CBCVA, 4) and analogous neutral cannabinoids and cannabinoid carboxylic acids, and other synthetic analogs.

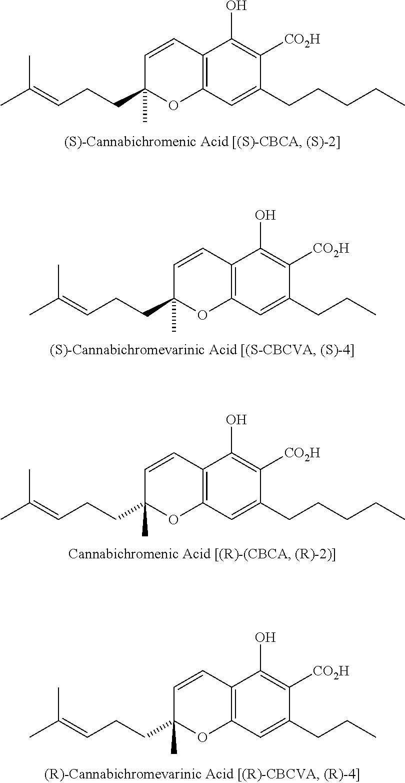

Protecting groups are well known to persons skilled in the art and are described in textbooks such as Greene and Wuts, (P. G. M. Wuts, T. W. Greene, “Greene's Protective Groups in Organic Synthesis”, 2006, Fourth Edition, John Wiley, New York).

Cleavage of the dioxinone rings of intermediate 9 by saponification or an equivalent process to produce the cannabinoid carboxylic acids 7 (R^{A}═CO₂H) is carried out as described in R. Cookson, T. N. Barrett and A. G. M. Barrett, “β-Keto-dioxinones and β,δ-Diketo-dioxinones in Biomimetic Resorcylate Total Synthesis”, Accounts of Chemical Research, 2015, volume 48, pages 628-642 and references therein. Decarboxylation of the cannabinoid carboxylic acids 7 (R^{A}═CO₂H) to provide cannabinoids 7 (R^{A}═H) is carried out as described in H. Perrotin-Brunel, W. Buijs, J. van Spronsen, M. J. E. van Roosmalen, C. J. Peters, R. Verpoorte and G.-J. Witkamp, “Decarboxylation of Δ⁹-tetrahydrocannabinol: Kinetics and molecular modeling”, Journal of Molecular Structure, 2011, volume 987, pages 67-73 and references therein.

Amide formation is carried out by activation of the carboxylic acid for example by formation of the N-hydroxysuccinimide ester and coupling with the corresponding amine, for example see Goto (Y. Goto, Y. Shima, S. Morimoto, Y. Shoyama, H. Murakami, A. Kusai and K. Nojima, “Determination of tetrahydrocannabinolic acid—carrier protein conjugate by matrix-assisted laser desorption/ionization mass spectrometry and antibody formation”, Organic Mass Spectrometry, 1994, volume 29, pages 668-671). Alternative amide coupling reagents include but are not limited to dicyclohexyl carbodiimide (DCC), di-iso-propyl carbodiimide (DIC), O-(7-azabenzotriazol-1-yl)-1,1,3,3-tetramethyluronium hexafluorophosphate (HATU), O-(benzotriazol-1-yl)-1,1,3,3-tetramethyluronium hexafluorophosphate (HBTU) and bromotri(pyrrolidino)phosphonium hexafluorophosphate (PyBrop) (E. Valeur and M. Bradley, “Amide bond formation: beyond the myth of coupling reagents”, Chemical Society Reviews, 2009, volume 38, pages 606-631).

The aforementioned novel cannabinoids with formulae 7, (S)-7 or (R)-7 or the scalemic mixtures of (S)-7 and (R)-7 above may be used as active compounds either alone or admixed in combination with known cannabinoids such as but not limited to Δ⁹-tetrahydrocannabinol (THC), tetrahydrocannabivarin (THBV), cannabidiol (CBD) or cannabidivarin (CBDV) or other drugs for the treatment of infections, pain, multiple sclerosis-related spasticity, nausea, epilepsy, Alzheimer's brain injury/concussion, cancer, glaucoma and retinal degeneration, disorders of immune-inflammation, lung injury or disease, liver injury or disease, kidney injury or disease, eye injury or disease, amongst other pathologies. In some embodiments, the said novel cannabinoids with formulae 7, (S)-7 or (R)-7 above either alone or admixed in combination with known cannabinoids such as but not limited to Δ⁹-tetrahydrocannabinol (THC), tetrahydrocannabivarin (THBV), cannabidiol (CBD) or cannabidivarin (CBDV) are formulated into pharmaceutical compositions in a suitable form for administration to a patient. Such formulations, in addition to the active cannabinoid or cannabinoids or other drugs in a combination therapeutic agent, contain pharmaceutically acceptable diluents and excipients, which may include binders such as lactose, starches, cellulose, sorbitol, polyethylene glycol or polyvinyl alcohol or other pharmaceutically acceptable oligosaccharides or polymers, disintegrants such as polyvinylpyrrolidone, carboxymethylcellulose or other pharmaceutically acceptable disintegrants, vehicles such as petrolatum, dimethyl sulfoxide, mineral oil, or in omega-3 oil-in-water nanoemulsions, or as complexes with cyclodextrins such as hydroxypropyl-beta-cyclodextrin, preservatives including antioxidants such as vitamin A, vitamin E, vitamin C, retinyl palmitate, cysteine, methionine, sodium citrate, citric acid, parabens or alternative pharmaceutically acceptable preservatives, antiadherents, lubricants and glidants such as magnesium stearate, stearic acid, talc, silica, pharmaceutically acceptable fats or oils, coatings such as cellulose ether hydroxypropyl methylcellulose, gelatin or other pharmaceutically acceptable coatings, flavors and fragrances such as but not limited to the volatile terpenes of Cannabis and citrus fruits and other pharmaceutically acceptable diluents or excipients. The aforementioned pharmaceutical compositions may be administrated to a patient by enteral administration for example as a pill, tablet or capsule, by sublingual administration for example as a tablet, strip, drops, spray, lozenge, effervescent tablet, intranasal administration for example as a spray or micronized powder, inhalation administration for example as a spray or micronized powder, rectal administration for example as a suppository or solution, by parenteral drug administration by intramuscular, subcutaneous or intravenous injection for example of a solution or by other known methods of clinical administration.

The oxidative cyclization is suitable for the synthesis of novel cannabinoids 7, (S)-7 or (R)-7 and these compounds are also part of the invention. The invention includes synthesis of the target cannabinoids as oils or crystalline derivatives, as appropriate, including solvates, hydrates and polymorphs. These novel racemic cannabinoids 7 have the formula:

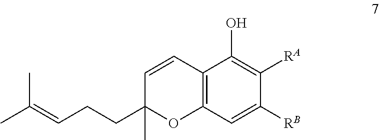

wherein:
   R^{A} is H;
   R^{B} is hydroxymethyl, 1-hydroxyethyl, 2-hydroxyethyl, 1,2-dihydroxyethyl or linear or branched C₃, C₄, C₆ to C₁₀ alkyl or double branched C₄, C₆ to C₁₀ alkyl in each case substituted by one or two hydroxyl groups or substituted by one or more fluoro-groups, (CH₂)ₚ—OR^{F};
   p is 1, 2, 3, 4, 5 or 6;
   R^{F} is C₁ to C₆ alkyl, (CH₂)ᵣ—C₃ to C₆ cycloalkyl;
   each r is independently 0, 1, 2, 3, 4, 5 or 6;
   with the exception that each hydroxyl group in R^{B} cannot be on the carbon bearing a fluoro group or a second hydroxyl group;
or
   R^{A} is CO₂H and its pharmaceutically acceptable salts or co-crystals, CO₂R^{C};
   R^{B} is hydroxymethyl, ethyl, 1-hydroxyethyl, 2-hydroxyethyl, 1,2-dihydroxyethyl, 1-fluoroethyl, 2-fluoroethyl; or linear or branched C₃ to C₅ alkyl substituted by one or two hydroxyl groups or substituted by one or more fluoro-groups; or linear or branched C₆ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{C} is C₁ to C₆ alkyl, (CH₂)_{q}—C₃ to C₆ cycloalkyl, allyl, optionally substituted benzyl or optionally substituted 2-phenylethyl;
   q is 0, 1, 2, 3, 4, 5 or 6;
   R^{F} is C₁ to C₆ alkyl, (CH₂)ᵣ—C₃ to C₆ cycloalkyl;
   each r is independently 0, 1, 2, 3, 4, 5 or 6;
   with the exception that each hydroxyl group in R^{B} cannot be on the carbon bearing a fluoro group or a second hydroxyl group;
or
   R^{A} is CONHR^{D}, CONR^{D}R^{E}
   R^{B} is H or C₁ to C₂ alkyl, linear or branched C₃ to C₁₀ alkyl or double branched C₄ to C₁₀ alkyl in each case optionally substituted by one or two hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{D} is C₁ to C₆ alkyl, (CH₂)ᵣ—C₃ to C₆ cycloalkyl, C₃ to C₆ cycloalkyl, allyl, optionally substituted benzyl or optionally substituted 2-phenylethyl; R^{E} is C₁ to C₆ alkyl, (CH₂)ᵣ—C₃ to C₆ cycloalkyl, allyl, optionally substituted benzyl or optionally substituted 2-phenylethyl; or NR^{D}R^{E} is azetidinyl, pyrrolidinyl, morpholinyl or piperidinyl each optionally substituted by one or two hydroxyl groups or one hydroxymethyl group with the exception that the hydroxyl groups cannot be on the carbon bearing the heterocyclic ring nitrogen or the heterocyclic ring oxygen with morpholine;
   R^{F} is C₁ to C₆ alkyl, (CH₂)ᵣ—C₃ to C₆ cycloalkyl;
   each r is independently 0, 1, 2, 3, 4, 5 or 6;
with the exception that each hydroxyl group in R^{B} cannot be on the carbon bearing a fluoro group or a second hydroxyl group.

The oxidative cyclization followed by resolution is suitable for the synthesis of novel single enantiomer cannabinoids (S)-7 and (R)-7 as well as scalemic mixtures of cannabinoids (S)-7 and (R)-7 and these enantiomers and scalemic mixtures and are also part of the invention. The invention includes synthesis of the target cannabinoids as oils or crystalline derivatives, as appropriate, including solvates, hydrates and polymorphs. These novel cannabinoids (S)-7 and (R)-7 have the formulae:

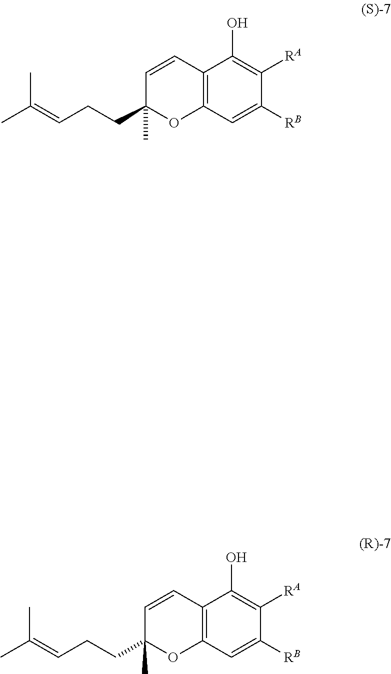

wherein:
   R^{A} is H;
   R^{B} is H, methyl, ethyl, n-butyl, n-hexyl, n-heptyl, n-octyl, n-nonyl or n-decyl in each case optionally substituted by one or two hydroxyl groups or optionally substituted by one or more fluoro-groups; or n-propyl or n-hexyl substituted by one or two hydroxyl groups or substituted by one or more fluoro-groups; or branched C₃ to C₁₀ alkyl or double branched C₄ to C₁₀ alkyl in each case optionally substituted by one or two hydroxyl groups or optionally substituted by one or more fluoro-groups; or (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{F} is C₁ to C₆ alkyl, (CH₂)ᵣ—C₃ to C₆ cycloalkyl;
   each r is independently 0, 1, 2, 3, 4, 5 or 6;
or
   R^{A} is CO₂H and its pharmaceutically acceptable salts or co-crystals, CO₂R^{C}, CONHR^{D}, CONR^{D}R^{E};
   R^{B} is H, methyl, ethyl, n-propyl, n-butyl, n-pentyl, n-hexyl, n-heptyl, n-octyl, n-nonyl or n-decyl in each case optionally substituted by one or two hydroxyl groups or optionally substituted by one or more fluoro-groups; or branched C₃ to C₁₀ alkyl or double branched C₄ to C₁₀ alkyl in each case optionally substituted by one or two hydroxyl groups or optionally substituted by one or more fluoro-groups; or (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{C} is C₁ to C₆ alkyl, (CH₂)_{q}—C₃ to C₆ cycloalkyl, allyl, optionally substituted benzyl or optionally substituted 2-phenylethyl;
   q is 0, 1, 2, 3, 4, 5 or 6;
   R^{D} is C₁ to C₆ alkyl, (CH₂)ᵣ—C₃ to C₆ cycloalkyl, C₃ to C₆ cycloalkyl, allyl, optionally substituted benzyl or optionally substituted 2-phenylethyl; R^{E} is C₁ to C₆ alkyl, (CH₂)ᵣ—C₃ to C₆ cycloalkyl, allyl, optionally substituted benzyl or optionally substituted 2-phenylethyl; or NR^{D}R^{E} is azetidinyl, pyrrolidinyl, morpholinyl or piperidinyl each optionally substituted by one or two hydroxyl groups or one hydroxymethyl group with the exception that the hydroxyl groups cannot be on the carbon bearing the heterocyclic ring nitrogen or the heterocyclic ring oxygen with morpholine;
   R^{F} is C₁ to C₆ alkyl, (CH₂)ᵣ—C₃ to C₆ cycloalkyl;
   each r is independently 0, 1, 2, 3, 4, 5 or 6;
with the exception that each hydroxyl group in R^{B} cannot be on the carbon bearing a fluoro group or a second hydroxyl group.

The oxidative cyclizations proceed via the intermediates 9 and these compounds are also part of the invention. These novel intermediates 9 have the formula:

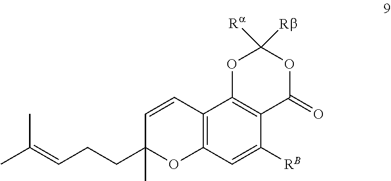

wherein:
   R^{B} is H, protected hydroxymethyl, or linear C₂ to C₁₀ alkyl or branched C₃ to C₁₀ alkyl or double branched C₄ to C₁₀ alkyl in each case optionally substituted by one or two protected hydroxyl groups or optionally substituted by one or more fluoro-groups, (CH₂)ₒ—C₃ to C₆ cycloalkyl, (CH₂)ₚ—OR^{F}, or C₃ to C₆ cycloalkyl optionally substituted by a C₁ to C₈ alkyl;
   o is 0, 1, 2, 3, 4, 5 or 6;
   p is 1, 2, 3, 4, 5 or 6;
   R^{F} is C₁ to C₆ alkyl, (CH₂)ᵣ—C₃ to C₆ cycloalkyl;
   Rα is C₁ to C₆ alkyl or optionally substituted aryl, and preferably methyl; and
   Rβ is C₁ to C₆ alkyl or optionally substituted aryl, and preferably methyl; or
   Rα and Rβ in combination are (CH₂)ₛ (s is 4, 5 or 6);
with the exception that each hydroxyl group in R^{B} cannot be on the carbon bearing a fluoro group or a second hydroxyl group.

### EXAMPLES

### (E)-3,7-Dimethylocta-2,6-dien-1-yl 4-(2,2-dimethyl-4-oxo-4H-1,3-dioxin-6-yl)-3-oxobutanoate

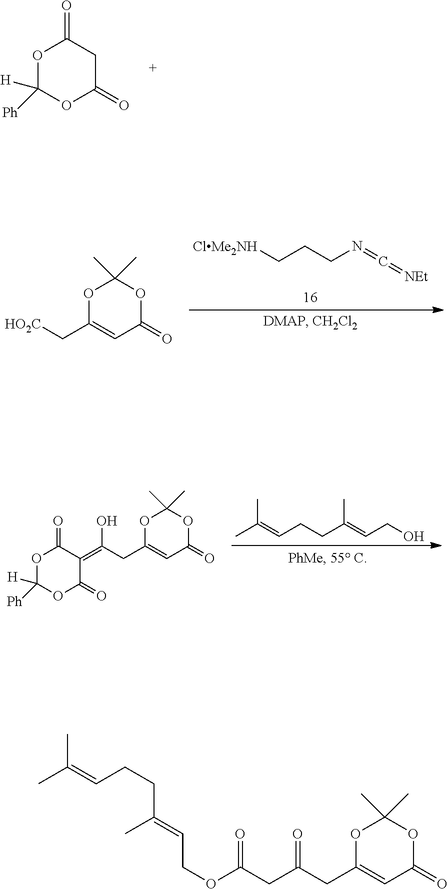

N-(3-Dimethylaminopropyl)-N′-ethylcarbodiimide hydrochloride (10 g, 50 mmol) and 4-dimethylaminopyridine (6.1 g, 50 mmol) were sequentially added to 2-phenyl-1,3-dioxane-4,6-dione (9.6 g, 50 mmol) in EtOAc (250 mL). After 5 min, 2-(2,2-dimethyl-4-oxo-4H-1,3-dioxin-6-yl)acetic acid (9.3 g, 50 mmol) was added. The reaction mixture was stirred for 2 h at room temperature. After this period, aqueous HCl (2M; 250 mL) was added, and the biphasic mixture was vigorously stirred for 10 min. The phases were separated, and the aqueous phase was extracted with EtOAc (250 mL). The combined EtOAc extracts was dried (MgSO₄), filtered, and concentrated under reduced pressure. The residue was immediately dissolved in anhydrous PhMe (250 mL) with vigorous stirring, and geraniol (4.3 mL, 25 mmol) was added dropwise. The solution was heated to 60° C. and, after 3 h and the starting material had been consumed, was concentrated under reduced pressure. The crude product was purified by flash column chromatography (EtOAc pentane; 1:10 to 1:5) to give (E)-3,7-dimethylocta-2,6-dien-1-yl 4-(2,2-dimethyl-4-oxo-4H-1,3-dioxin-6-yl)-3-oxobutanoate (8.1 g, 22 mmol, 88%) as a colorless oil: ¹H NMR (400 MHz, CDCl₃) δ 5.43-5.27 (m, 2H), 5.11-5.04 (m, 1H), 4.71-4.63 (m, 2H), 3.51 (s, 2H), 3.50 (d, J=0.5 Hz, 2H), 2.15-2.00 (m, 4H), 1.71 (s, 6H), 1.69 (s, 1H), 1.68 (d, J=1.3 Hz, 4H), 1.60 (d, J=1.4 Hz, 3H); ¹³C NMR (101 MHz, CDCl₃) δ 195.8, 166.5, 163.7, 143.8, 132.1, 123.7, 117.4, 107.5, 97.3, 62.8, 49.3, 47.1, 39.7, 26.4, 25.8, 25.2, 17.9, 16.7; IR (neat) 2966, 2917, 2856, 1718, 1636, 1388, 1270, 1200, 1014, 900 cm⁻¹; HRMS (ES+) m/z calc for C₂₀H₂₉O₆ [M+H]⁺ 365.1959, found 365.1968.

### (E)-8-(3,7-Dimethylocta-2,6-dien-1-yl)-7-hydroxy-2,2-dimethyl-5-pentyl-4H-benzo[d][1,3]dioxin-4-one

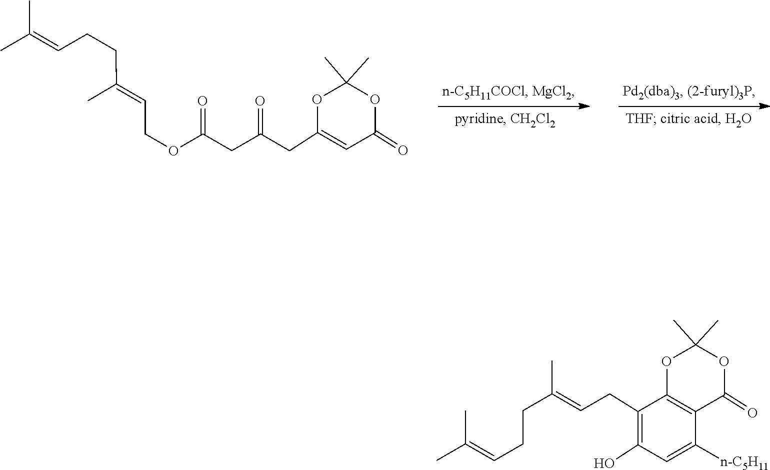

MgCl₂ (0.95 g, 10 mmol) was added with stirring to (E)-3,7-dimethylocta-2,6-dien-1-yl 4-(2,2-dimethyl-4-oxo-4H-1,3-dioxin-6-yl)-3-oxobutanoate (3.6 g, 10 mmol) in anhydrous MeCN (50 mL) and the mixture cooled to 0° C. Pyridine (1.6 mL, 20 mmol) was added dropwise with stirring and, after 5 min, n-hexanoyl chloride (1.4 mL, 10 mmol) was added dropwise. After 1 h at 0° C., saturated aqueous NH₄Cl (50 mL) was added, and the mixture was acidified to pH 1 with aqueous HCl (1M; 4 mL). The biphasic mixture was separated, and the aqueous phase was extracted with EtOAc (2×100 mL). The combined organic phases were washed with brine (200 mL), dried (MgSO₄), filtered, and concentrated under reduced pressure. The residual oil was immediately dissolved in anhydrous THF (50 mL) and tri(2-furyl)phosphine (116 mg, 0.5 mmol) and tris(dibenzylideneacetone)dipalladium(0) (116 mg, 0.126 mmol) were added sequentially with stirring. After 1 h, Et₃N (4.2 mL, 30 mmol) was added dropwise with stirring, and, after 13 h, reaction was quenched with 10% aqueous citric acid (100 mL). The biphasic mixture was separated, and the aqueous layer was extracted with EtOAc (3×100 mL) and the combined organic extracts was dried (MgSO₄), filtered, and concentrated under reduced pressure. The residue was purified by flash column chromatography (Et₂O pentane 1:10) to provide (E)-8-(3,7-dimethylocta-2,6-dien-1-yl)-7-hydroxy-2,2-dimethyl-5-pentyl-4H-benzo[d][1,3]dioxin-4-one (1.6 g, 4 mmol, 40%) as a pale-yellow solid: ¹H NMR (400 MHz, CDCl₃) δ 6.42 (s, 1H), 6.10 (s, 1H), 5.24-5.14 (m, 1H), 5.04 (dddd, J=7, 5.5, 3.5, 1.5 Hz, 1H), 3.32 (dd, J=7, 1 Hz, 2H), 3.04-2.94 (m, 2H), 2.16-1.99 (m, 5H), 1.79 (d, J=1.3 Hz, 3H), 1.67 (s, 7H), 1.75-1.52 (m, 6H), 1.66 (d, J=1.5 Hz, 3H), 1.58 (d, J=1.5 Hz, 3H), 1.34 (tq, J=5, 3 Hz, 5H), 0.93-0.82 (m, 3H); ¹³C NMR (101 MHz, CDCl₃) δ 160.6, 160.1, 156.1, 147.8, 138.6, 132.0, 123.7, 120.9, 112.9, 112.7, 105.0, 104.6, 39.7, 34.3, 31.9, 30.6, 26.4, 25.7, 22.6, 22.0, 17.7, 16.2, 14.1; IR (neat) 3291 (br), 2956, 2924, 2855, 1690, 1605, 1590, 1293, 1276, 1208, 1165, 1113, 1047 cm⁻¹; HRMS (ES+) m/z calc for C₂₅H₃₇O₄ [M+H]⁺ 401.2686, found 401.2686.

### 2,2,8-Trimethyl-8-(4-methylpent-3-en-1-yl)-5-pentyl-4H,8H-[1,3]dioxino[4,5-f]chromen-4-one

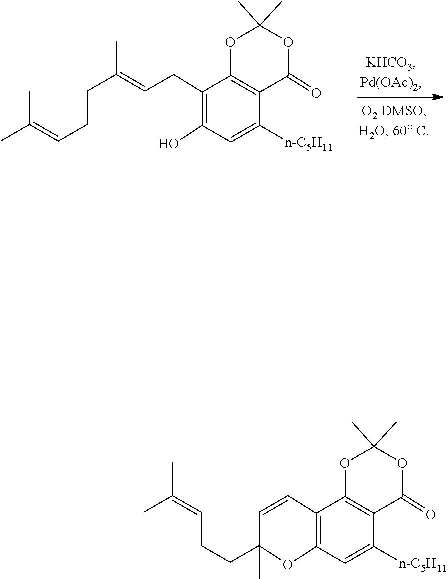

KHCO₃ (440 mg, 4.4 mmol) and Pd(OAc)₂ (90 mg, 0.40 mmol) were added with stirring to an O₂ saturated solution of (E)-8-(3,7-dimethylocta-2,6-dien-1-yl)-7-hydroxy-2,2-dimethyl-5-pentyl-4H-benzo[d][1,3]dioxin-4-one (1.6 g, 4 mmol) in DMSO (100 mL) and H₂O (10 mL). The reaction mixture was heated at 60° C. for 18 h under an atmosphere of O₂ (1 atm). The mixture was diluted with Et₂O (50 mL), and aqueous HCl was added (1M; 50 mL). The biphasic mixture was separated, and the aqueous phase was extract with Et₂O (3×50 mL). The combined organic phases were washed with H₂O (50 mL), dried (MgSO₄), filtered, and concentrated under reduced pressure. The crude product was purified by flash column chromatography (Et₂O:pentane 1:10) to give 2,2,8-trimethyl-8-(4-methylpent-3-en-1-yl)-5-pentyl-4H,8H-[1,3]dioxino[4,5-f]chromen-4-one (970 mg, 2.44 mmol, 61%) as a colorless oil: ¹H NMR (400 MHz, CDCl₃) δ 6.54 (dd, J=10.1, 0.7 Hz, 1H), 6.37 (s, 1H), 5.52 (d, J=10.1 Hz, 1H), 5.08 (ddq, J=8.5, 7, 1.5 Hz, 1H), 3.04-2.92 (m, 2H), 2.16-2.02 (m, 2H), 1.82-1.60 (m, 4H), 1.75 (m, 1H), 1.69 (s, 6H), 1.65 (d, J=1.3 Hz, 3H), 1.56 (d, J=1.3 Hz, 3H), 1.41 (s, 3H), 1.38-1.29 (m, 4H), 0.92-0.85 (m, 3H); ¹³C NMR (101 MHz, CDCl₃) δ 160.3, 158.9, 153.1, 150.0, 132.1, 127.9, 123.9, 116.0, 113.1, 107.5, 104.9, 104.5, 80.2, 41.7, 34.7, 32.0, 30.6, 27.19, 25.9, 25.9, 25.8, 22.8, 22.7, 17.8, 14.2; IR (neat) 2921, 2956, 2856, 1726, 1563, 1387, 1280, 1150, 1129, 1037 cm⁻¹; HRMS (ES+) m/z calc for C₂₅H₃₄O₄ [M+H]+ 393.2535, found 393.2558.

### Cannabichromene

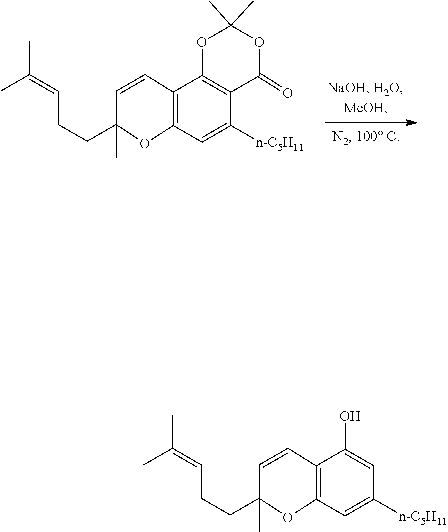

Aqueous NaOH (6M; 5 mL), purged with nitrogen using three freeze-pump-thaw cycles, was added dropwise stirring to 2,2,8-trimethyl-8-(4-methylpent-3-en-1-yl)-5-pentyl-4H,8H-[1,3]dioxino[4,5-f]chromen-4-one (400 mg, 1 mmol) in MeOH (5 mL), and the mixture was heated at 100° C. for 16 h. The mixture was cooled to room temperature, diluted with Et₂O (10 mL), and acidified using 10% aqueous citric acid (10 mL). After 5 min, the biphasic solution was separated, and the aqueous layer was extracted with Et₂O (3×10 mL). The combined organic extracts were washed with brine (30 mL), dried (MgSO₄), filtered, and concentrated under reduced pressure. The residue was purified by flash column chromatography (Et₂O pentane 1:10) to give cannabichromene (220 mg, 0.7 mmol, 70%) as an orange oil: ¹H NMR (400 MHz, CDCl₃) δ 6.63 (dd, J=10.0, 0.8 Hz, 1H), 6.28 (s, 1H), 6.14 (d, J=1.5 Hz, 1H), 5.52 (d, J=10.0 Hz, 1H), 5.12 (tdq, J=7.2, 2.9, 1.4 Hz, 1H), 4.68 (s, 1H), 2.46 (t, J=7.8 Hz, 2H), 2.19-2.06 (m, 3H), 1.79-1.52 (m, 8H), 1.39-1.24 (m, 4H), 0.91 (t, J=6.6 Hz, 3H); ¹³C NMR (101 MHz, CDCl₃) δ 154.2, 151.1, 144.9, 131.8, 127.4, 124.3, 116.9, 109.3, 107.8, 107.1, 78.3, 41.2, 36.0, 31.6, 30.8, 30.5, 26.4, 25.8, 22.9, 22.7, 17.8, 14.2; IR (neat) 3410, 2958, 2924, 2855, 1622, 1576, 1429, 1142 1082, 1051 cm⁻¹; HRMS (ES+) m/z calc for C₂₁H₃₁O₂ [M+H]⁺ 315.2324, found 315.2325.

CBC HPLC Conditions (Analytical - 1 mg of CBC)

| Column | Chiralpak IG-U 100 × 3 mm 1.6 um | |
|---|---|---|
| Mobile Phase | A:B 45:55 | |
| A | 0.1% HCOOH in Water | |
| B | 0.1% HCOOH in MeCN | |
| Gradient | Isocratic | |
| Flowrate | 0.6 | mL/min |
| Temperature | 25° C. (controlled) | |
| Injection | 3 | μL |
| volume | | |

| | |
|---|---|
| Detection | VWD or DAD at 220 nm without |
| | reference |
| Sample | 1:1 Water:MeCN, 0.1 mg/ml |

CBC HPLC Conditions (Preparative - 4 mg of CBC)

| Column | Chiralpak IG-U 100 × 3 mm 1.6 um | |
|---|---|---|
| Mobile Phase | A:B 35:65 | |
| A | 0.1% HCOOH in Water | |
| B | 0.1% HCOOH in MeCN | |
| Gradient | Isocratic | |
| Flowrate | 1.0 | mL/min |
| Temperature | 25° C. (controlled) | |
| Injection | 20 | μL |
| volume | | |

| | |
|---|---|
| Detection | VWD or DAD at 220 nm without |
| | reference |
| Sample | 1:1 Water:MeCN |

### Cannabichromenic Acid

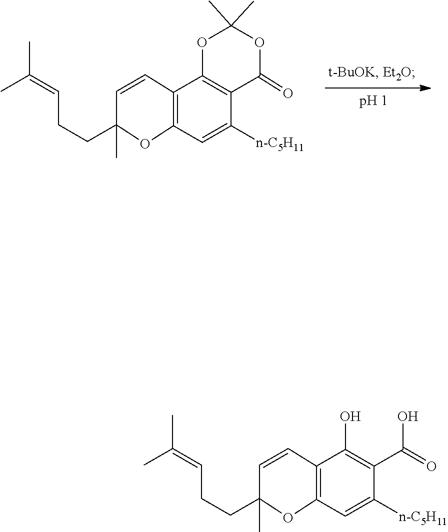

t-BuOK (450 mg, 4 mmol) was suspended in Et₂O (5 mL), and 2,2,8-trimethyl-8-(4-methylpent-3-en-1-yl)-5-pentyl-4H,8H-[1,3]dioxino[4,5-f]chromen-4-one (200 mg, 0.5 mmol) in Et₂O (1 mL) was added with stirring. After 72 h, H₂O (10 mL) and Et₂O (10 mL) were added, and the biphasic mixture was separated. The organic layer was extracted with water (5×10 mL) and the combined aqueous solutions were acidified with aqueous 4M HCl to pH 1. The acidic solution was extracted with EtOAc (5×10 mL). The combined organic extracts were dried (MgSO₄), filtered, and concentrated under reduced pressure. The residue was purified by column chromatography (EtOAc:pentane 1:6) to give cannabichromenic acid (102 mg, 0.28 mmol, 57%) as a colorless oil: ¹H NMR (400 MHz, CDCl₃) δ 11.75 (s, 1H), 6.78-6.71 (m, 1H), 6.25 (s, 1H), 5.48 (d, J=10.1 Hz, 1H), 5.09 (dddd, J=8.6, 5.7, 2.8, 1.4 Hz, 1H), 2.92-2.85 (m, 2H), 2.09 (q, J=7.9 Hz, 2H), 1.82-1.69 (m, 1H), 1.69-1.58 (m, 3H), 1.66 (d, J=1.4 Hz, 3H), 1.57 (d, J=1.3 Hz, 3H), 1.41 (s, 3H), 1.39-1.29 (m, 4H), 0.91 (td, J=7.0, 5.9, 2.8 Hz, 3H); ¹³C NMR (101 MHz, CDCl₃) δ 176.0, 160.8, 159.1, 149.6, 132.0, 126.5, 124.0, 116.8, 111.7, 107.2, 103.0, 80.2, 41.8, 36.9, 32.2, 31.5, 27.3, 25.8, 22.8, 22.6, 17.8, 14.2; IR (neat) 3310 (v. broad), 2958, 2924, 2858, 1615, 1565, 1453, 1259 1171 cm-1; HRMS (ES−) m/z calc for C₂₂H₂₉O₄ [M−H]⁺ 357.2066, found 357.2074.

1. A racemic compound of structure 7: 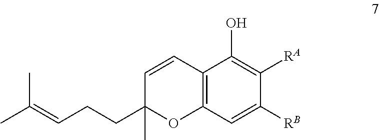
wherein:
R^{A} is H;
R^{B} is selected from the group consisting of hydroxymethyl, 1-hydroxyethyl, 2-hydroxyethyl, 1,2-dihydroxyethyl, or linear or branched C₃, C₄, C₆ to C₁₀ alkyl or double branched C₄, C₆ to C₁₀ alkyl in each case substituted by one or two hydroxyl groups or substituted by one or more fluoro-groups, (CH₂)ₚ—OR^{F};
p is selected from 1, 2, 3, 4, 5 and 6;
R^{F} is selected from the group consisting of C₁ to C₆ alkyl, (CH₂)ᵣ—C₃ to C₆ cycloalkyl; and
r is selected from 0, 1, 2, 3, 4, 5 and 6,
with the proviso that each hydroxyl group in R^{B} cannot be on the carbon bearing a fluoro group or a second hydroxyl group;
or
R^{A} is CO₂H and its pharmaceutically acceptable salts or co-crystals, CO₂R^{C};
R^{B} is selected from the group consisting of hydroxymethyl, ethyl, 1-hydroxyethyl, 2-hydroxyethyl, 1,2-dihydroxyethyl, 1-fluoroethyl, 2-fluoroethyl, linear or branched C₃ to C₅ alkyl substituted by one or two hydroxyl groups or substituted by one or more fluoro-groups, linear or branched C₆ to C₁₀ alkyl optionally substituted by one or two hydroxyl groups or optionally substituted by one or more fluoro-groups, double branched C₄ to C₁₀ alkyl optionally substituted by one or two hydroxyl groups or optionally substituted by one or more fluoro-groups, (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{C} is selected from the group consisting of C₁ to C₆ alkyl, (CH₂)_{q}—C₃ to C₆ cycloalkyl, allyl, optionally substituted benzyl and optionally substituted 2-phenylethyl;
q is selected from 0, 1, 2, 3, 4, 5 and 6;
R^{F} is C₁ to C₆ alkyl or (CH₂)ᵣ—C₃ to C₆ cycloalkyl; and
r is selected from 0, 1, 2, 3, 4, 5 and 6,
with the proviso that each hydroxyl group in R^{B} cannot be on the carbon bearing a fluoro group or a second hydroxyl group;
or
R^{A} is CONHR^{D} or CONR^{D}R^{E};
R^{B} is selected from H, C₁ to C₂ alkyl optionally substituted by one or two hydroxyl groups or optionally substituted by one or more fluoro-groups, linear or branched C₃ to C₁₀ alkyl optionally substituted by one or two hydroxyl groups or optionally substituted by one or more fluoro-groups, double branched C₄ to C₁₀ alkyl optionally substituted by one or two hydroxyl groups or optionally substituted by one or more fluoro-groups, (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{D} is selected from C₁ to C₆ alkyl, (CH₂)ᵣ—C₃ to C₆ cycloalkyl, C₃ to C₆ cycloalkyl, allyl, optionally substituted benzyl, optionally substituted 2-phenylethyl; and R^{E} is selected from C₁ to C₆ alkyl, (CH₂)ᵣ—C₃ to C₆ cycloalkyl, allyl, optionally substituted benzyl, optionally substituted 2-phenylethyl; or
NR^{D}R^{E} is selected from azetidinyl, pyrrolidinyl, morpholinyl and piperidinyl, each optionally substituted by one or two hydroxyl groups or one hydroxymethyl group with the proviso that the hydroxyl groups cannot be on the carbon bearing the heterocyclic ring nitrogen or the heterocyclic ring oxygen with morpholine;
R^{F} is C₁ to C₆ alkyl or (CH₂)ᵣ—C₃ to C₆ cycloalkyl; and
r is selected from 0, 1, 2, 3, 4, 5 and 6,
with the proviso that each hydroxyl group in R^{B} cannot be on the carbon bearing a fluoro group or a second hydroxyl group.

2. The racemic compound of claim 1, wherein:
R^{A} is H; and
R^{B} is selected from the group consisting of hydroxymethyl, 1-hydroxyethyl, 2-hydroxyethyl, 1,2-dihydroxyethyl, linear or branched C₃—, C₄—, or C₆-to-C₁₀ alkyl substituted by one or two hydroxyl groups, and double branched C₄—, or C₆-to-C₁₀ alkyl substituted by one or two hydroxyl groups.

3. The racemic compound of claim 1, wherein:
R^{A} is CO₂H and its pharmaceutically acceptable salts or co-crystals;
R^{B} is selected from the group consisting of hydroxymethyl, ethyl, 1-hydroxyethyl, 2-hydroxyethyl, 1,2-dihydroxyethyl, 1-fluoroethyl, 2-fluoroethyl, linear or branched C₃ to C₅ alkyl substituted by one or two hydroxyl groups or substituted by one or more fluoro-groups, linear or branched C₆ to C₁₀ alkyl optionally substituted by one or two hydroxyl groups or optionally substituted by one or more fluoro-groups, double branched C₄ to C₁₀ alkyl optionally substituted by one or two hydroxyl groups or optionally substituted by one or more fluoro-groups, (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{F} is C₁ to C₆ alkyl or (CH₂)ᵣ—C₃ to C₆ cycloalkyl; and
r is selected from 0, 1, 2, 3, 4, 5 and 6.

4. The racemic compound of claim 1, wherein:
R^{A} is CO₂R^{C};
R^{B} is selected from the group consisting of hydroxymethyl, ethyl, 1-hydroxyethyl, 2-hydroxyethyl, 1,2-dihydroxyethyl, 1-fluoroethyl, 2-fluoroethyl, linear or branched C₃ to C₅ alkyl substituted by one or two hydroxyl groups or substituted by one or more fluoro-groups, linear or branched C₆ to C₁₀ alkyl optionally substituted by one or two hydroxyl groups or optionally substituted by one or more fluoro-groups, double branched C₄ to C₁₀ alkyl optionally substituted by one or two hydroxyl groups or optionally substituted by one or more fluoro-groups, (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{C} is selected from the group consisting of C₁ to C₆ alkyl, (CH₂)_{q}—C₃ to C₆ cycloalkyl, allyl, optionally substituted benzyl and optionally substituted 2-phenylethyl;
q is selected from 0, 1, 2, 3, 4, 5 and 6;
R^{F} is C₁ to C₆ alkyl or (CH₂)ᵣ—C₃ to C₆ cycloalkyl; and
r is selected from 0, 1, 2, 3, 4, 5 and 6.

5. The racemic compound of claim 1, wherein:
R^{A} is CONHR^{D} or CONR^{D}R^{E};
R^{B} is selected from H, C₁ to C₂ alkyl optionally substituted by one or two hydroxyl groups or optionally substituted by one or more fluoro-groups, linear or branched C₃ to C₁₀ alkyl optionally substituted by one or two hydroxyl groups or optionally substituted by one or more fluoro-groups, double branched C₄ to C₁₀ alkyl optionally substituted by one or two hydroxyl groups or optionally substituted by one or more fluoro-groups, (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{D} is selected from C₁ to C₆ alkyl, (CH₂)ᵣ—C₃ to C₆ cycloalkyl, C₃ to C₆ cycloalkyl, allyl, optionally substituted benzyl, optionally substituted 2-phenylethyl; and R^{E} is selected from C₁ to C₆ alkyl, (CH₂)ᵣ—C₃ to C₆ cycloalkyl, allyl, optionally substituted benzyl, optionally substituted 2-phenylethyl; or
NR^{D}R^{E} is selected from azetidinyl, pyrrolidinyl, morpholinyl and piperidinyl, each optionally substituted by one or two hydroxyl groups or one hydroxymethyl group, with the proviso that the hydroxyl groups cannot be on the carbon bearing the heterocyclic ring nitrogen or the heterocyclic ring oxygen with morpholine;
R^{F} is C₁ to C₆ alkyl or (CH₂)ᵣ—C₃ to C₆ cycloalkyl; and
r is selected from 0, 1, 2, 3, 4, 5 and 6.

6. A single enantiomer compound of structure (S)-7 or (R)-7 or a scalemic mixture thereof: 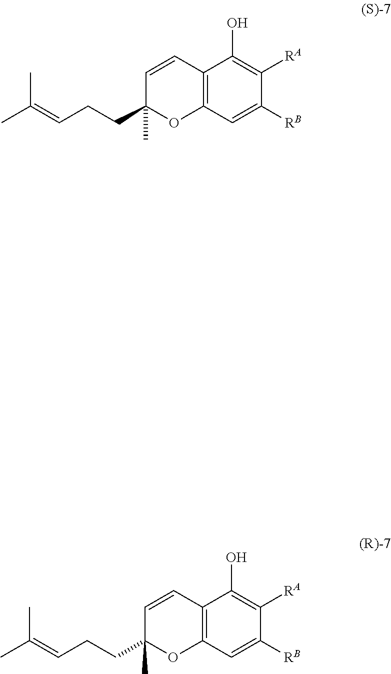
wherein:
R^{A} is H;
R^{B} is selected from the group consisting of H, methyl, ethyl, n-butyl, n-hexyl, n-heptyl, n-octyl, n-nonyl and n-decyl, in each case optionally substituted by one or two hydroxyl groups or optionally substituted by one or more fluoro-groups;
n-propyl or n-hexyl substituted by one or two hydroxyl groups or substituted by one or more fluoro-groups; branched C₃ to C₁₀ alkyl optionally substituted by one or two hydroxyl groups or optionally substituted by one or more fluoro-groups or double branched C₄ to C₁₀ alkyl substituted by one or two hydroxyl groups or optionally substituted by one or more fluoro-groups; (CH₂)ₒ—C₃ to C₆ cycloalkyl, (CH₂)ₚ—OR^{F}, and C₃ to C₆ cycloalkyl, optionally substituted by a C₁ to C₈ alkyl;
o is selected from 0, 1, 2, 3, 4, 5 and 6;
p is selected from 1, 2, 3, 4, 5 and 6;
R^{F} is C₁ to C₆ alkyl or (CH₂)ᵣ—C₃ to C₆ cycloalkyl;
r is selected from 0, 1, 2, 3, 4, 5 and 6;
or
R^{A} is selected from the group consisting of CO₂H and its pharmaceutically acceptable salts or co-crystals, CO₂R^{C}, CONHR^{D}, and CONR^{D}R^{E};
R^{B} is selected from the group consisting of H, methyl, ethyl, n-propyl, n-butyl, n-pentyl, n-hexyl, n-heptyl, n-octyl, n-nonyl or n-decyl, in each case optionally substituted by one or two hydroxyl groups or optionally substituted by one or more fluoro-groups; branched C₃ to C₁₀ alkyl or double branched C₄ to C₁₀ alkyl, in each case optionally substituted by one or two hydroxyl groups or optionally substituted by one or more fluoro-groups; (CH₂)ₒ—C₃ to C₆ cycloalkyl, (CH₂)ₚ—OR^{F}, and C₃ to C₆ cycloalkyl, optionally substituted by a C₁ to C₈ alkyl;
o is selected from 0, 1, 2, 3, 4, 5 and 6;
p is selected from 1, 2, 3, 4, 5 and 6;
R^{C} is selected from the group consisting of C₁ to C₆ alkyl, (CH₂)_{q}—C₃ to C₆ cycloalkyl, allyl, optionally substituted benzyl and optionally substituted 2-phenylethyl;
q is selected from 0, 1, 2, 3, 4, 5 and 6;
R^{D} is selected from the group consisting of C₁ to C₆ alkyl, (CH₂)ᵣ—C₃ to C₆ cycloalkyl, C₃ to C₆ cycloalkyl, allyl, optionally substituted benzyl, optionally substituted 2-phenylethyl; and R^{E} is selected from the group consisting of C₁ to C₆ alkyl, (CH₂)ᵣ—C₃ to C₆ cycloalkyl, allyl, optionally substituted benzyl and optionally substituted 2-phenylethyl; or NR^{D}R^{E} is selected from the group consisting of azetidinyl, pyrrolidinyl, morpholinyl and piperidinyl, each optionally substituted by one or two hydroxyl groups or one hydroxymethyl group, with the proviso that the hydroxyl groups cannot be on the carbon bearing the heterocyclic ring nitrogen or the heterocyclic ring oxygen with morpholine;
R^{F} is C₁ to C₆ alkyl or (CH₂)ᵣ—C₃ to C₆ cycloalkyl; and
r is selected from 0, 1, 2, 3, 4, 5 and 6,
with the proviso that each hydroxyl group in R^{B} cannot be on the carbon bearing a fluoro group or a second hydroxyl group.

7. The single enantiomer compound with structure (S)-7 or (R)-7 or a scalemic mixture thereof of claim 6, wherein:
R^{A} is H;
R^{B} is selected from the group consisting of H, methyl, ethyl, n-butyl, n-hexyl, n-heptyl, n-octyl, n-nonyl or n-decyl in each case optionally substituted by one or two hydroxyl groups or optionally substituted by one or more fluoro-groups; or n-propyl or n-hexyl substituted by one or two hydroxyl groups or substituted by one or more fluoro-groups; branched C₃ to C₁₀ alkyl or double branched C₄ to C₁₀ alkyl, in each case optionally substituted by one or two hydroxyl groups or optionally substituted by one or more fluoro-groups; (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{F} is C₁ to C₆ alkyl or (CH₂)ᵣ—C₃ to C₆ cycloalkyl; and
r is selected from 0, 1, 2, 3, 4, 5 and 6.

8. The single enantiomer compound with structure (S)-7 or (R)-7 or a scalemic mixture thereof of claim 6, wherein:
R^{A} is CO₂H and its pharmaceutically acceptable salts or co-crystals;
R^{B} is selected from the group consisting of H, methyl, ethyl, n-propyl, n-butyl, n-pentyl, n-hexyl, n-heptyl, n-octyl, n-nonyl, n-decyl, in each case optionally substituted by one or two hydroxyl groups or optionally substituted by one or more fluoro-groups; branched C₃ to C₁₀ alkyl or double branched C₄ to C₁₀ alkyl, in each case optionally substituted by one or two hydroxyl groups or optionally substituted by one or more fluoro-groups; (CH₂)ₒ—C₃ to C₆ cycloalkyl, (CH₂)ₚ—OR^{F}, and C₃ to C₆ cycloalkyl, optionally substituted by a C₁ to C₈ alkyl;
o is selected from 0, 1, 2, 3, 4, 5 and 6;
p selected from 1, 2, 3, 4, 5 and 6;
R^{F} is C₁ to C₆ alkyl or (CH₂)ᵣ—C₃ to C₆ cycloalkyl; and
r is 0, 1, 2, 3, 4, 5 and 6.

9. The single enantiomer compound with structure (S)-7 or (R)-7 or a scalemic mixture thereof of claim 6, wherein:
R^{A} is CO₂R^{C};
R^{B} is selected from the group consisting of H, methyl, ethyl, n-propyl, n-butyl, n-pentyl, n-hexyl, n-heptyl, n-octyl, n-nonyl, n-decyl, in each case optionally substituted by one or two hydroxyl groups or optionally substituted by one or more fluoro-groups; branched C₃ to C₁₀ alkyl or double branched C₄ to C₁₀ alkyl, in each case optionally substituted by one or two hydroxyl groups or optionally substituted by one or more fluoro-groups; (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{C} is selected from the group consisting of C₁ to C₆ alkyl, (CH₂)_{q}—C₃ to C₆ cycloalkyl, allyl, optionally substituted benzyl and optionally substituted 2-phenylethyl;
q is selected from 0, 1, 2, 3, 4, 5 and 6;
R^{F} is C₁ to C₆ alkyl or (CH₂)ᵣ—C₃ to C₆ cycloalkyl; and
r is selected from 0, 1, 2, 3, 4, 5 and 6.

10. The single enantiomer compound with structure (S)-7 or (R)-7 or a scalemic mixture thereof of claim 6:
wherein:
R^{A} is CONR^{D}R^{E};
R^{B} is selected from the group consisting of H, methyl, ethyl, n-propyl, n-butyl, n-pentyl, n-hexyl, n-heptyl, n-octyl, n-nonyl, n-decyl, in each case optionally substituted by one or two hydroxyl groups or optionally substituted by one or more fluoro-groups; branched C₃ to C₁₀ alkyl or double branched C₄ to C₁₀ alkyl, in each case optionally substituted by one or two hydroxyl groups or optionally substituted by one or more fluoro-groups; (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{D} is selected from the group consisting of C₁ to C₆ alkyl, (CH₂)ᵣ—C₃ to C₆ cycloalkyl, C₃ to C₆ cycloalkyl, allyl, optionally substituted benzyl, optionally substituted 2-phenylethyl; and R^{E} is selected from the group consisting of C₁ to C₆ alkyl, (CH₂)ᵣ—C₃ to C₆ cycloalkyl, allyl, optionally substituted benzyl or optionally substituted 2-phenylethyl; or NR^{D}R^{E} is selected from the group consisting of azetidinyl, pyrrolidinyl, morpholinyl and piperidinyl, each optionally substituted by one or two hydroxyl groups or one hydroxymethyl group with the proviso that the hydroxyl groups cannot be on the carbon bearing the heterocyclic ring nitrogen or the heterocyclic ring oxygen with morpholine;
R^{F} is C₁ to C₆ alkyl or (CH₂)ᵣ—C₃ to C₆ cycloalkyl; and
r is selected from 0, 1, 2, 3, 4, 5 and 6.

11. An intermediate compound of structure 9: 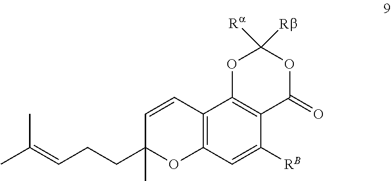
wherein:
R^{B} is selected from the group consisting of H, protected hydroxymethyl, linear C₂ to C₁₀ alkyl optionally substituted by one or two protected hydroxyl groups or optionally substituted by one or more fluoro-groups, branched C₃ to C₁₀ alkyl optionally substituted by one or two protected hydroxyl groups or optionally substituted by one or more fluoro-groups, double branched C₄ to C₁₀ alkyl optionally substituted by one or two protected hydroxyl groups or optionally substituted by one or more fluoro-groups, (CH₂)ₒ—C₃ to C₆ cycloalkyl, (CH₂)ₚ—OR^{F}, and C₃ to C₆ cycloalkyl optionally substituted by a C₁ to C₈ alkyl;
o is selected from 0, 1, 2, 3, 4, 5 and 6;
p is selected from 1, 2, 3, 4, 5 and 6;
R^{F} is C₁ to C₆ alkyl or (CH₂)ᵣ—C₃ to C₆ cycloalkyl;
Rα is C₁ to C₆ alkyl or optionally substituted aryl; and
Rβ is C₁ to C₆ alkyl or optionally substituted aryl;
with the proviso that each protected hydroxyl group in R^{B} cannot be on the carbon bearing a fluoro group or a second protected hydroxyl group.

12. The intermediate compound of claim 11, wherein:
Rα is methyl; and
Rβ is methyl.

13-20. (canceled)
